(19) United States
(12) Patent Application Publication
(10) Pub. No.: US 2025/0297235 A1
(43) Pub. Date: Sep. 25, 2025

(54) VARIANTS OF THE HUMAN ATM PROTEIN FOR THE TREATMENT OF DISEASES RELATED TO AT LEAST ONE MUTATION OF THE ATM GENE

(71) Applicant: Quince Therapeutics S.P.A., Bresso (IT)

(72) Inventors: Michele Menotta, Urbino (IT); Anastasia Ricci, Urbino (IT); Federica Biancucci, Urbino (IT); Mauro Magnani, Urbino (IT)

(73) Assignee: Quince Therapeutics S.P.A., Bresso (IT)

(21) Appl. No.: 18/838,105

(22) PCT Filed: Feb. 9, 2023
(86) PCT No.: PCT/IB2023/051155

§ 371 Date: Aug. 13, 2024

(30) Foreign Application Priority Data
Feb. 14, 2022 (IT) 102022000002645

(51) Int. Cl. C12N 9/12; A61K 38/45; C12N 15/86

(52) U.S. Cl. CPC C12N 9/1205; A61K 38/45; C12N 15/86; C12Y 207/11001; C12N 2740/15043

(57) ABSTRACT

The present invention relates to variants of the human ATM protein or derivatives thereof, said variant and/or derivatives for use in the treatment or in the prevention of diseases related to at least one mutation of the ATM gene, i.e. diseases caused or induced by said mutation/s, mRNAs and cDNAs, expression vectors coding for said variant of the ATM protein or derivatives thereof and composition or associations comprising them.

### CROSS-REFERENCE TO RELATED PATENT APPLICATIONS

This application is a U.S. National Phase Application under 35 U.S.C. § 371 of International Application No. PCT/IB2023/051155, filed on Feb. 9, 2023, which claims the benefit of and priority to Italian Patent Application No. 102022000002645, filed on Feb. 14, 2022, the contents of which are incorporated herein by reference in their entirety.

### BACKGROUND

The present invention relates to variants of the human ATM protein or derivatives thereof, said variant and/or derivatives for use in the treatment or in the prevention of diseases related to at least one mutation of the ATM gene, i.e. diseases caused or induced by said mutation/s, mRNAs and cDNAs, expression vectors coding for said variant of the ATM protein or derivatives thereof and composition or associations comprising them.

### STATE OF THE ART

Ataxia Telangiectasia (AT) is a very rare neurodegenerative disease caused by biallelic mutations in the Ataxia Telangiectasia Mutated (ATM) gene, which codes a protein of the same name ATM with several substrates. No therapy is currently available for these patients.

Monoallelic mutations of the ATM gene are also well known in the art for being related to a number of cancers and to be strictly related to the increase of cancer risk in subjects carrying said mutations both in the germ line as well as in the somatic cells line.

The ATM gene causing the AT disorder was discovered by Savitsky K, et al. The gene, which codes for the protein of the same name ATM, a member of the Pl3 kinase-like kinase (Pl3KK) family, is transcribed into 27 different mRNAs and into 20 known alternative splicing mRNA.

However, the occasionally discover of positive effects of dexamethasone on neurological symptoms of AT patients led to a new hope for a plausible treatment. Dexamethasone 25 was able to partly restore ATM activity in AT lymphoblastoid cells by a new ATM transcript, ‘ATMdexa1’, originated from alternative splicing of ATM messenger, that can be translated into a functional protein, named ‘miniATM’. ATMdexal and other ATM variants have also been identified in vivo in the blood of AT patients.

ATM variants were capable of rescuing ATM activity in AT cells, particularly in the nuclear 30 role of DNA double-strand breaks (DSBs) recognition and repair, and in the cytoplasmic role of modulating autophagy, antioxidant capacity and mitochondria functionality, all features essential for post-mitotic neurons survival. These outcomes are trigged by the kinase and the additional domains of the tested ATM variants. They are useful to restore cellular functionality and they are applicable in gene therapy or gene delivery for the treatment of AT or the treatment of somatic pathology caused by ATM malfunction, like for example some type of malignancies.

In the last years, it has been found that treatment with glucocorticoid analogues could improve the neurological symptoms of AT patients 1, but the steroid side effects such us gain weight and moon face, occurred in most of patients and the therapy must be interrupt. To overcome the adverse effects of long-term treatment with oral steroids a new approach has been considered for AT treatment: the use of red blood cells as drug delivery. EryDel SpA (Bresso, Milano, Italy), a drug delivery company, proposed the EryDex system (Dexamethasone Sodium Phosphate delivered through autologous red blood cells) for the treatment of AT, leading to a slow release of the active drug (dexamethasone) up to one month in the bloodstream, improving its efficacy with reduced toxicity. Dexamethasone (dex) was chosen since its high anti-inflammatory potency especially on central nervous system, without mineralocorticoid activity. The improvement was more evident in patients that have milder neurological symptoms and in patients that were good responder to the drug loading in their erythrocytes. The molecular mechanism involved index action is still unknown. In 2012, Menotta et al. tried to give a possible explanation of dex positive effects in AT. They found that treatment with dex in vitro could restore ATM activity in AT lymphoblastoid cells by a new ATM transcript originating from a non-canonical splicing. This transcript ‘ATMdexa1’ can be translated into a functional protein with reduced activity, named ‘miniATM’ of 252 amino acids and a molecular weight of 28 kDa. The mini protein was not detected in WT cells, MiniATM maintains the kinase domain of native ATM and partially rescue ATM deficiency. ‘ATMdexa1’ has also been identified in vivo in the blood of AT patients treated with intra-erythrocyte Dexamethasone, while it was not detected in untreated AT patients and healthy subjects. The expression of ‘ATMdexa1’ depends on the treatment and correlates with a positive response to dex therapy. In these patients it was also possible to isolate new ‘ATMdexa1’ variants, originating from canonical (exons 3-52, 4-53and 2-52) and non-canonical (short direct repeats: 3-52 and 4-51) splicing of the ATM mRNA, each containing the same coding sequence identified in ‘ATMdexa1’ and some more domains. They were observed in different patients and in the same patient at different time point, and even more than one variant was present in the same sample.

It has been previously reported by researchers that the introduction of wild type ATM cDNA with HSV amplicon vector could restore some functions in AT human fibroblasts, and its injection into AT mouse cerebella would lead to a retain ATM expression. However, vectors used for this purpose are not entirely safe and since they are not integrative, the transduction of the protein results transient.

Carranza et al. constructed a lentiviral vector containing a full-length ATM capable to rescue AT deficiencies in repairing radiation-induced DSBs and regaining radio-sensitivity. However, the vector has a very low transduction efficiency and is therefore not suitable for an effective and optimal therapy of AT deficiencies.

Therefore, the challenge is to find ATM variants that are effective in restoring deficiencies caused by ATM mutations that are more efficient with respect to mini ATM or to vectors coding for full ATM, to treat AT patients.

### SUMMARY OF THE INVENTION

The Authors of the present invention, in order to provide alternatives and more effective therapies for diseases caused by ATM mutations, investigated whether fragments of ATM cDNAs coding for at least the terminal P13K domain and additional domains could provide an improved restoration of the phenotype caused by ATM mutations with respect to the miniATM (SEQ ID NO 4) described in the state of the art as the identification of a small, yet effective, molecule providing suitable candidates for efficient transduction with respect to the full length ATM (SEQ ID NO 5). The authors of the present invention therefore investigated whether it was possible to provide ATM variants with improved effectiveness with respect to miniATM. A number of variants were isolated, (a non limiting example is provided by variants 3-52 of SEQ ID NO 2 and 4-53 SEQ ID NO 3), however, although sharing the miniATM sequence in combination with additional domains of the full-length human ATM, said variants did not provide an improved effectiveness with respect to miniATM, as reported in table 1.

The authors hence, also named as ATM SINT in the present description and figures, designed synthetic ATM variants, among which ATM variant having SEQ ID NO 1, achieved a very high transduction efficiency in fibroblasts (almost 100% of the cells resulted transduced), i.e., an improved efficiency with respect to the whole ATM gene thereby overcoming the cargo limit of the actual vectors approved for gene therapy and provided an improved effectiveness in restoring the phenotype caused by ATM mutations with respect to miniATM. Therefore, the present invention provides a new ATM variant with improved performances with respect to the miniATM disclosed in the state of the art, able to regain ATM roles due to additional domains than in miniATM, and a most efficient transduction of cells.

ATM variant of the invention could also be successfully administered by using nanoparticles or vesicles delivery, that is less immunogenic, less expensive, and more efficient in crossing the blood-brain barrier. In fact, the Authors of the present invention have demonstrated that ATM variants such as the ones described herein have the ability in bypassing the DNA DSBs and the other ATM extra-nuclear biochemical functions including autophagy and mitochondrial activity.

In other words, the Authors of the present invention were able to provide an in silico designed variant having SEQ ID NO 1, that is capable to restore most of the wild-type ATM functions i.e. repairing of DNA DSBs, autophagy progression, better mitochondria functionality and capacity to alter HDAC4 localization promoting HDAC4 nuclear export. In addition, due to its small size, said variant is also suitable for overcoming the cargo limit of the actual vectors approved for gene therapy. Other natural variants analysed by the inventors, including the mini-ATM are less efficient in restoring the above described wild-type ATM functions, as demonstrated in the figures and in table 1 below.

TABLE 1
A summary table with all the tested activities of AT transduced cells is reported
(see figures and examples), showing the activity of miniATM, already verified
by Menotta et al. and showing the enhanced effects of ATM SINT.

| | | | | | | Oxidative | | |
|---|---|---|---|---|---|---|---|---|
| | | | | Autophagy | ER Stress | stress | Mitochondria | HDAC4 |
| | yH2AX | p-CHK2 | p-p53 | recovery | recovery | recovery | recovery | Shuttle |
| WT | ++ | ++ | ++ | ++ | ++ | ++ | ++ | ++ |
| UTD | −ATR | −ATR | −ATR | − | − | − | − | − |
| | crosstalk | crosstalk | crosstalk | | | | | |
| TD 3-52 | +− | + | + | + | ++ | − | + | + |
| TD 4-53 | +− | +− | + | + | ++ | + | + | + |
| TD SINT | ++ | + | + | + | ++ | − | ++ | ++ |
| TD | +− | +− | + | +− | ++ | ++ | +− | + |
| miniATM | | | | | | | | |

yH2AX: recognition and repair of DNA DSBs.
p-CHK2/p-p53: biomarker of ATM activity in response to DNA damage, involved in the cell cycle arrest ensuring that the DNA is repaired before cell cycle progression.
Autophagy recovery: autophagy progression. This pathway is impaired in A-T cells.
Oxidative stress recovery: antioxidant capacity, ability to counteract ROS production.
Mitochondria recovery: less depolarized damaged mitochondria probably due to a better mitochondria functionality or to a greater mitophagy flux.
HDAC4 Shuttle: capacity to alter HDAC4 localization promoting HDAC4nuclear export, which is one of the protective mechanisms against neurodegeneration.

In all parts of the description ATM SINT is the ATM variant having SEQ ID NO 1, miniATM is the variant having SEQ ID NO 4, ATM 3-52 is an ATM variant having SEQ ID NO 2, ATM 4-53 is an ATM variant having SEQ ID NO 3 and wild-type human ATM has SEQ ID NO 5.

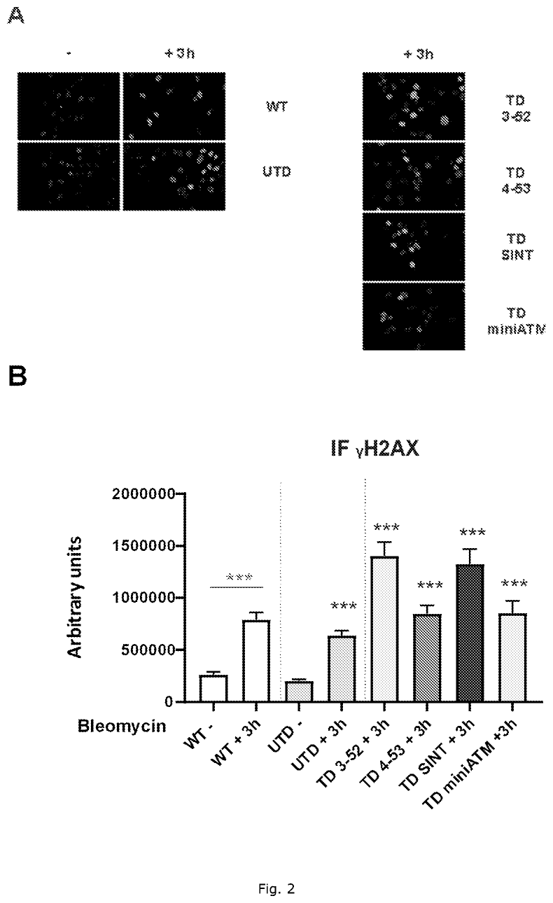

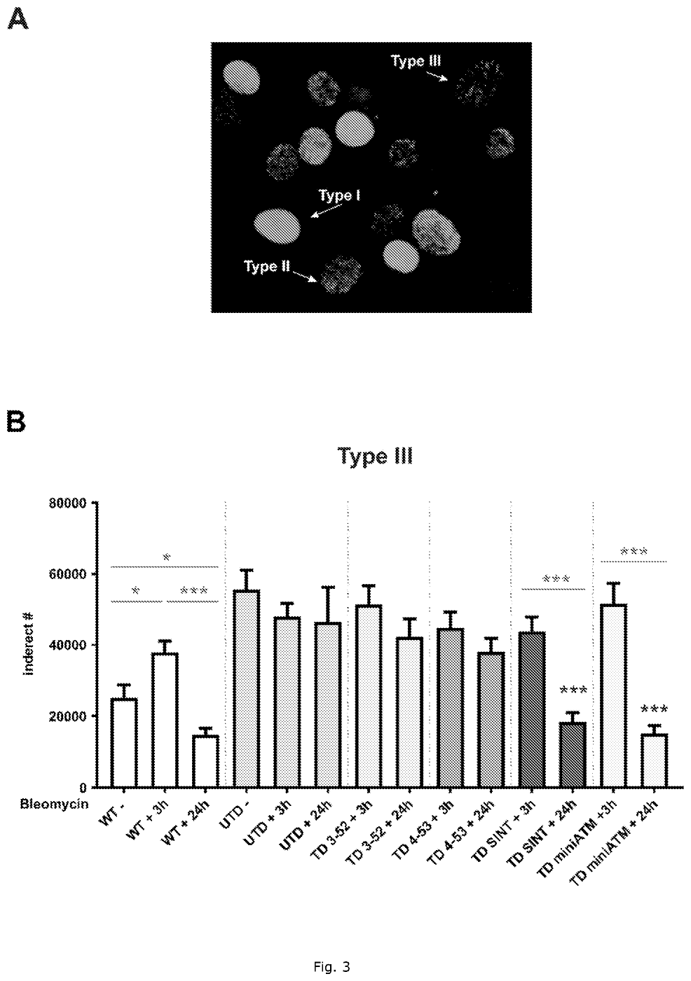

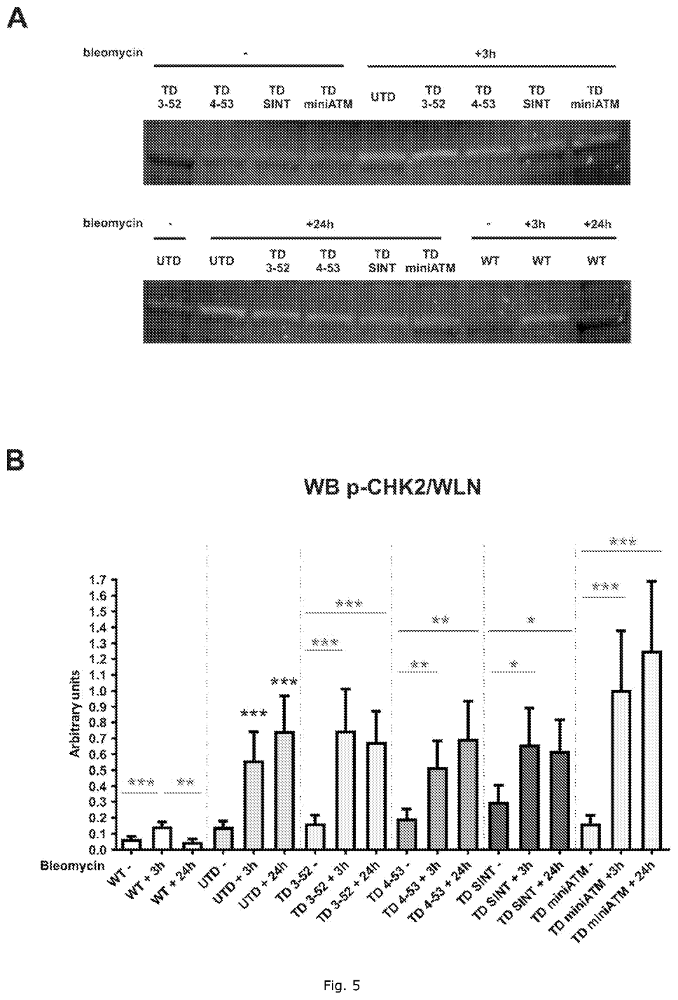

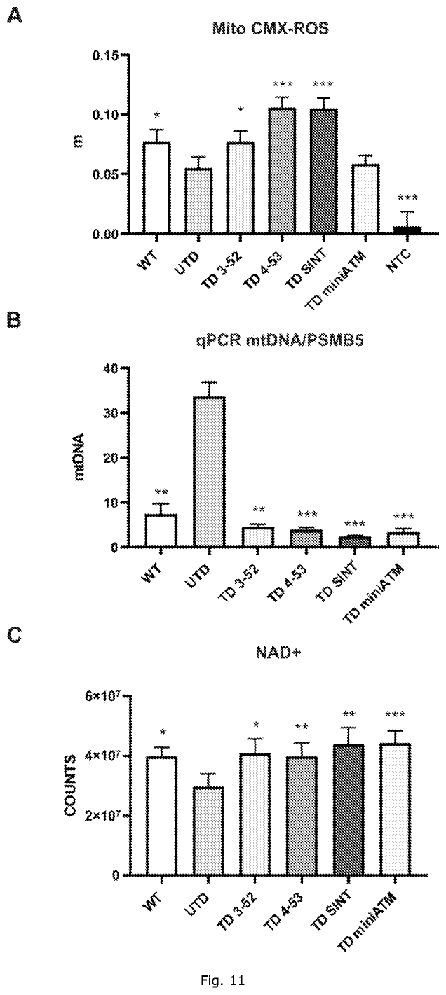

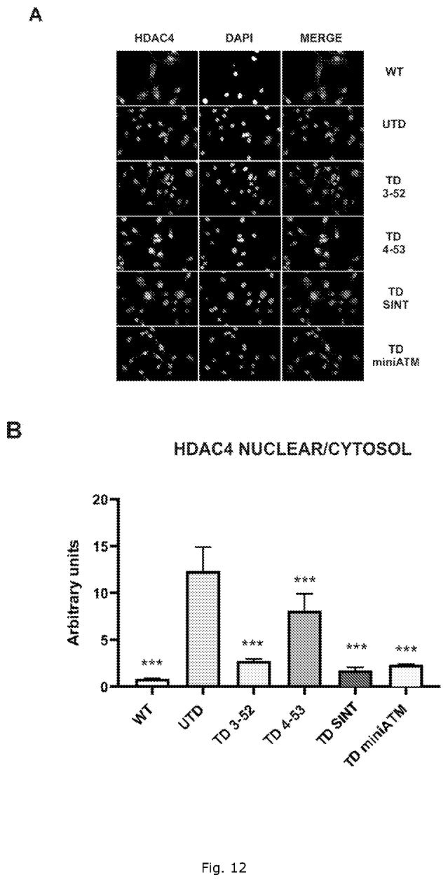

As depicted in FIG. 2, ATM SINT is capable of promoting H2AX phosphorylation, which is activated in the repairing of the DNA double-strand breaks (DSBs). Furthermore, variant ATM SINT is capable to statistically decrease Type III foci during the recovery, suggesting that most of the lesions were reduced in the cell line tested with said variant, as happened in Wild type cells (FIG. 3). Similarly, miniATM statistically reduced the Type III foci number at the same time condition, however miniATM is the less capable in phosphorylating H2AX as clear from the figures and from Table 1. ATM 3-52 (SEQ ID NO 2) and ATM 4-53 (SEQ ID NO 3) are capable to phosphorylate H2AX, but their repair process occurred slower than ATM SINT. Additionally, in the quantification of p-CHK2/WLN (FIG. 5), a biomarker of ATM activity involved in the cell cycle arrest when DNA lesion are present, only ATM 3-52 and ATM SINT show a reduction of CHK2 phosphorylation 24 h post drug, as in Wild-Type cells. Furthermore, ATM 4-53 and ATM SINT showed a greater mitochondria potential than ATM 3-52 in mitochondria functionality when compared to the other tested variants (FIG. 11). This feature was assayed since ATM is activated in response to mitochondria dysfunctions and an ATM loss of function leads to a reduced mitochondria membrane potential and impaired mitophagy. Finally, ATM SINT is the most efficient variant in reducing accumulation of HDAC4 (FIG. 12). This function was assayed since ATM is indirectly implicated in HDAC4 nuclear export, and an ATM loss of function leads to HDAC4 nuclear import and neurodegeneration in AT neurons.

Therefore, objects of the present invention are:
A variant of the human ATM protein having SEQ ID NO 1, or a derivative thereof;
A nucleotide sequence coding for the variant of the human ATM protein having SEQ ID NO 1 or for a derivative thereof as herein defined;
An expression vector comprising a nucleotide sequence coding for the variant of the human ATM protein having SEQ ID NO 1, or a derivative thereof operably linked to a promoter;
A pharmaceutical composition comprising the variant of the human ATM protein as defined in the description or in the claims, or the mRNA according as defined in the description or in the claims, or the expression vector as defined in the description or in the claims, and a pharmaceutically acceptable carrier and/or excipient;

The use of the variant of the human ATM protein or a derivative thereof as defined in the description or in the claims, or of the mRNA as defined in the description or in the claims, or of the nucleotide sequence as defined in the description or in the claims, or of the expression vector as defined in the description or in the claims, or of the pharmaceutical composition as defined in the description or in the claims, or of the combination as defined in the description or in the claims, as a medicament, in particular in the prevention or in the treatment in adjuvating the treatment of a disease related to at least one mutation of the ATM gene/s.

A further object of the invention is a method for the treatment, for adjuvating a treatment or for the prevention of diseases related to at least one mutation of the ATM gene/s, wherein the variant of the human ATM protein as defined in the description or in the claims, or the mRNA according as defined in the description or in the claims, or the nucleotide sequence as defined in the description or in the claims, or the expression vector as defined in the description or in the claims or the pharmaceutical composition as defined in the description or in the claims, or of the combination as defined in the description or in the claims, is administered in therapeutically effective amount to a subject in need thereof, optionally in combination with a therapeutic compound or with a therapeutic treatment.

According to the invention, diseases related to at least one mutation of the ATM gene/s are, diseases in which one or more mutation of the ATM gene is the direct cause of a disease or is an ascertained co-cause of a disease. Monoallelic as well as biallelic mutations of the ATM genes are known in the art to cause or co-cause a large number of diseases. In conformity with the state of the art said diseases include Ataxia Telangiectasia as well all cancer forms in which the mutation of the ATM gene/s in the affected subject results in a malfunction or loss of function of the ATM protein.

Additional advantages and/or embodiments of the present invention will be evident from the following detailed description.

### Glossary

Gene therapy according to the present description has the meaning commonly recognized in the art, it therefore refers to a therapy through transfer of genetic material (e.g., replacing a mutated gene with a healthy copy, or inactivating a mutated gene functioning improperly, or introducing a new gene, such as a gene coding for a therapeutic protein according to the invention, into the body) in the subject in need of a treatment, i.e., the therapeutic delivery of nucleic acid into a patient's cells as a drug to treat disease. Gene therapy according to the art and to the present invention can be achieved by transferring the genetic material of interest in the subject in need of treatment using a mRNA molecule or a non-viral or a viral method. Viral expression vectors commonly used for human gene therapy include retroviruses, adenoviruses, lentiviruses, herpes simplex virus, vaccinia virus, and adeno-associated virus. Viral vector genomes are either incorporated in the host's genome or stay as episomes. A mRNA molecule according to the present description is a mRNA molecule suitable for gene therapy, i.e., a molecule comprising 3′ and 5′ UTR elements flanking the coding sequence, a 5′ Cap and a polyA tail.

In any part of the description and of the claims, the expression “for use” in a treatment encompasses also the use of one or more therapeutic proteins (including isoforms or homologous), constructs, vectors, mRNAas defined in the description or in the claims for the preparation of a medicament for said treatment, wherein said one or more variants (in the form of therapeutic proteins), constructs, vectors, mRNAs are formulated with one or more suitable excipients and/or carriers into a medicament that can be administered for the treatment of diseases related to the mutation of the ATM gene as illustrated in the present description.

In any part of the present description and claims the expression “disease related to the mutation of the ATM gene” can be substituted by the expression “disease in a patient having monoallelic or biallelic mutation/s of the ATM gene”.

By biallelic mutation it is intended that both ATM genes are mutated, by monoallelic mutation it is intended that only one ATM gene is mutated.

In the present description the expression “diseases related to at least one mutation of the ATM gene/s” refers to diseases in which one or more mutation of the ATM gene is a direct cause of a disease or is an ascertained co-cause of a disease in a subject carrying said mutation. In other words, “diseases related to at least one mutation of the ATM gene/s” refers to diseases affecting subjects in which a mutation in the ATM gene/s results in a malfunction or loss of function of the ATM protein coded by said gene/s. In any part of the present description and claims the term “comprising” can be substituted by the term “consisting of”.

In all parts of the description ATM SINT is the ATM variant having SEQ ID NO 1, miniATM is the variant having SEQ ID NO 4, ATM 3-52 is an ATM variant having SEQ ID NO 2, ATM 4-53 is an ATM variant having SEQ ID NO 3 and wild-type human ATM has SEQ ID NO 5.

### DETAILED DESCRIPTION OF THE DRAWINGS

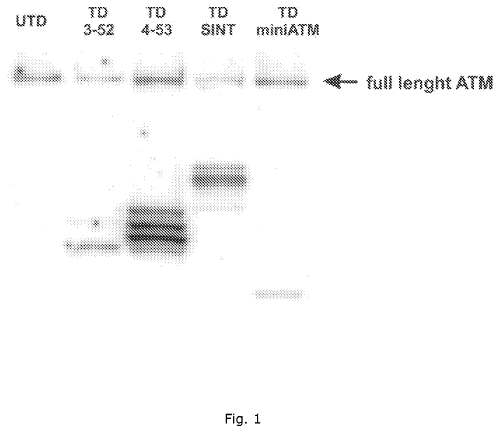

FIG. 1. Western Blot of AT 648 hT fibroblasts expressing ATM 3-52, ATM 4-53, ATM SINT, miniATM and untransduced AT 648 hT fibroblast. The arrow indicates the native mutated full-length ATM.

FIG. 2. IF quantification of H2AX phosphorylation (yH2AX) of WT hT and 648 hT transduced and untransduced cells treated with bleomycin for 3 h. Bleomycin was used to induce the DNA DSBs. When the DNA damage occurs, H2AX is phosphorylated by ATM and forms nuclear foci at the site of DNA DSBs. All transduced cells and WT hT enhanced yH2AX in response to bleomycin in comparison with UTD-. Accordingly, the capacity of the ATM variants to promote H2AX phosphorylation suggested that ATM variants were activated in response to the DNA damage, especially ATM SINT and 3-52, and they were able to recognize the DNA DSBs. At least 300 nuclei were counted for data processing. Red asterisks refer to intra sample comparison, while the black ones refer to statistical comparison with the control cell line UTD-(Kruskal Wallis followed by Dunn's test). Graphs show Mean with SEM.

FIG. 3. Quantitation of Type III foci of WT hT and 648 hT transduced and untransduced cells treated with bleomycin for 3 h and then kept in recovery course for 24 h. Assessed the capacity of the ATM variants to become activated in response to DNA damage, we proceeded to investigate their role in the repair process evaluating the reduction of Type III foci 24 h post treatment. The correction of the lesions depends on the reduction of Type III foci, rather than the reduction of the fluorescence intensity since H2AX phosphorylation could decrease 24 h post drug preserving the same number of foci, indicating the incomplete success of the DNA repair/the failure to completely repair the DNA lesions. The figure shows that only ATM SINT is able to statistically decrease Type III foci during the recovery, suggesting that most of the lesions were reduced in this cell line, as happened in WT hT cells. Similarly, miniATM statistically reduced the Type III foci number at the same time condition, but miniATM was the least capable in phosphorylating H2AX (see FIG. 2). Concerning ATM 3-52 and ATM 4-53 functions, we can assess that they were able to phosphorylate H2AX, but their repair process occurred slower than ATM SINT. UTD cells kept high number of foci, independently of bleomycin treatment, implying the persistence of unrepaired DNA lesions, since no ATM activity is present to counteract the DNA damage. Red asterisks refer to intra sample comparison, while the black ones show the statistical comparison with the control cell line UTD-. (Welch's ANOVA test). Graphs show Mean with SEM.

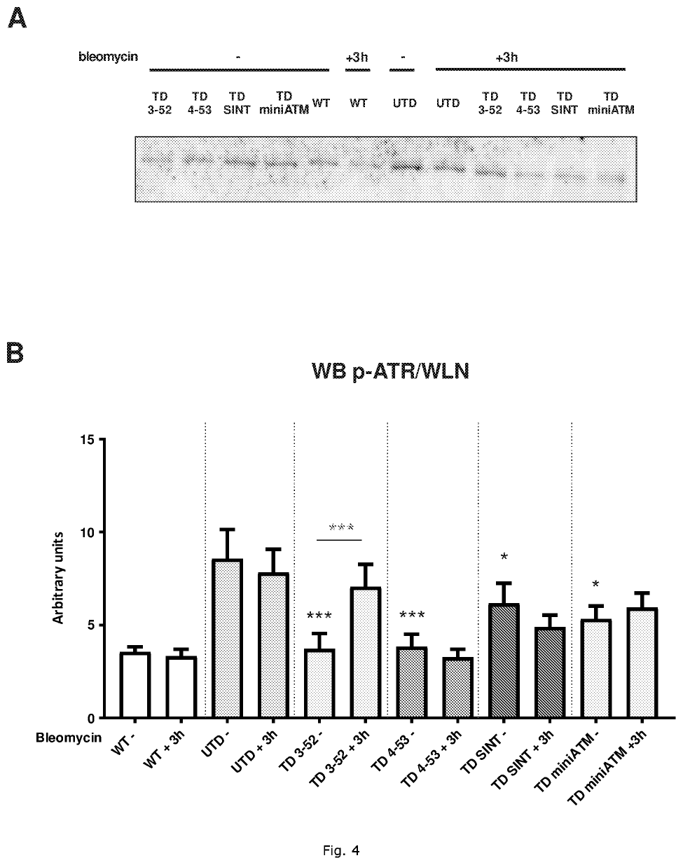

FIG. 4. WB quantification of p-ATR/WLN ratio in all the tested cells treated with bleomycin for 3 h. ATR phosphorylation was evaluated to justify the H2AX phosphorylation at 3 h post drug in UTD cells, observed in FIG. 2. It is known that H2AX could be phosphorylated by ATR in response to replication stress, and the absence of ATM could lead to a replication stress condition. Accordingly, the high content of phosphorylated ATR compared to ATM variants at basal condition suggests a replication stress in the absence of ATM in UTD cells. This condition persisted even after 3 h of drug treatment, indicating that UTD cells did not respond to the bleomycin treatment. Red asterisks refer to intra sample comparison, while the black ones refer to the statistical comparison with the control cell line UTD-(Friedman test followed by Dunn's test). Graphs show Mean with SEM.

FIG. 5. WB quantification of p-CHK2/WLN in all the tested samples, treated with bleomycin for 3 h and then kept in recovery course for 24 hours. The phosphorylation at Thr68 of CHK2 3-4 h post bleomycin is a biomarker of ATM activity since CHK2 is a target of ATM, involved in the cell cycle arrest when DNA lesions are present. On the contrary, its persistence after 24 h post drug indicates the non-completely activation of CHK2, since other phosphorylation sites are involved in its activation after the initial phosphorylation by ATM, justifying its analysis also at the recovery course. All transduce cells could phosphorylate CHK2 after 3 h of bleomycin treatment, demonstrating the functionality of ATM variants to correctly phosphorylate ATM substrates in response to DNA damage. Only ATM 3-52 and ATM SINT showed a reduction of CHK2 phosphorylation 24 h post drug, as happened in WT cells, despite not in a significant way, indicating an effort to mimic WT behaviour to completely activate CHK2. The phosphorylation of CHK2 in UTD cells is due to the crosstalk of ATM/ATR pathways in the absence of ATM. Red asterisks refer to intra sample comparison, while the black ones refer to statistical comparison with the control cell line UTD-. (Friedman test followed by Dunn's test). Graphs show Mean with SEM.

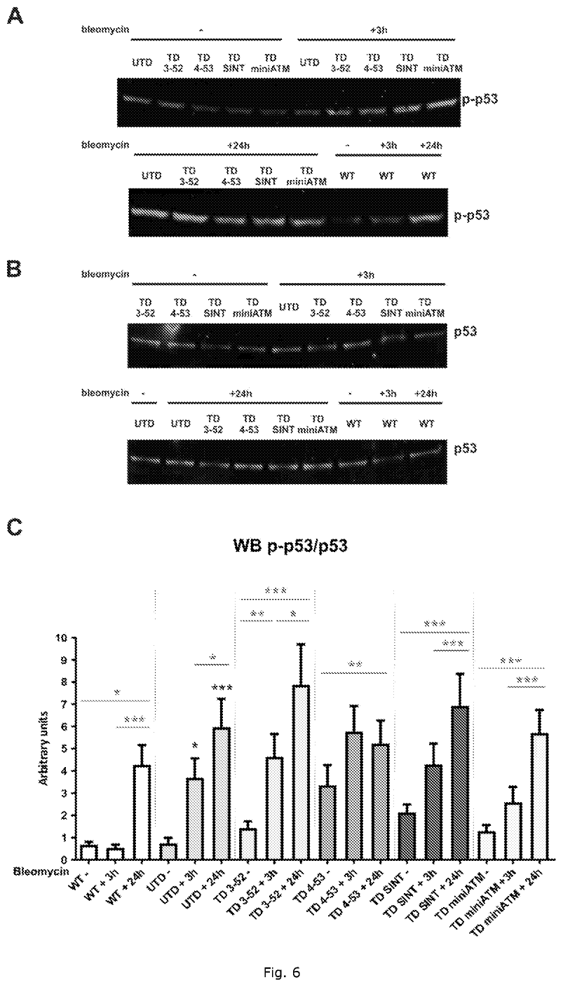

FIG. 6. WB quantification of p-53/p53 in all the tested samples, treated with bleomycin for 3h and then kept in recovery course for 24 hours. p53 is phosphorylated by ATM ensuring cell cycle arrest when DNA damage occurs. All transduced cells phosphorylate p53 in response to DNA damage, demonstrating the functionality of ATM variants to correctly phosphorylate ATM substrates. p53 phosphorylation was evaluated at 24 h post drug since its phosphorylation occurred slower than CHK2, probably because p53 is also a downstream target of CHK2 and its phosphorylation is required to support rather than start the cell cycle arrest. The phosphorylation of p53 in UTD cells is due to the crosstalk of ATM/ATR pathways in the absence of ATM. Red asterisks refer to intra sample comparison, while the black ones refer to statistical comparison with the control cell line UTD-. (Friedman test followed by Dunn's test). Graphs show Mean with SEM.

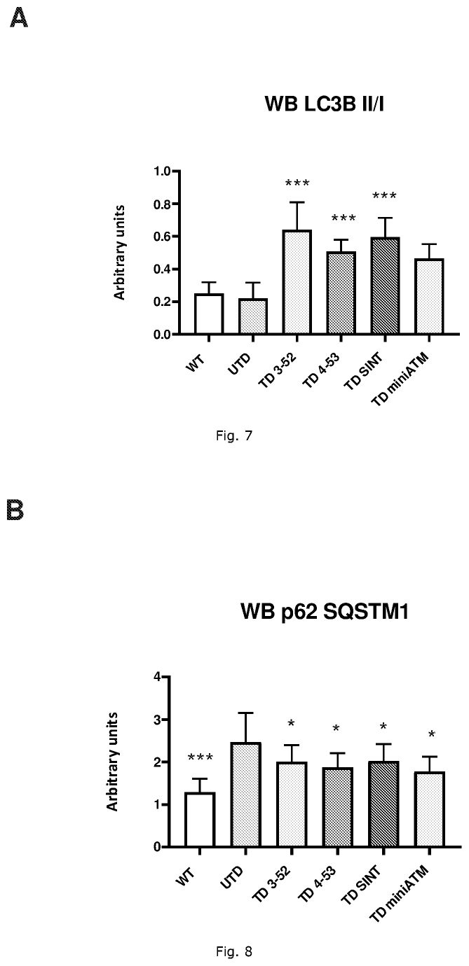

FIG. 7. WB quantification of LC3B II/I in all tested cell lines. LC3B is used as an autophagy marker to evaluate the autophagy progression since ATM also has a cytoplasmic role in indirectly promote the autophagy flux, whereas AT cells have an impairment in the fusion between autophagosomes and lysosomes.

Since the lipidated form LC3B II is associated with autophagosomes, the ratio LC3B II/1 is essential to evaluate the conversion of the LC3B Ito the lipidated LC3B II, indicating the progression of the autophagy flux. All ATM variants were able to increase the ratio of LC3B II/I, except for miniATM, in comparison with UTD cells, since they were able to promote the conversion of LC3B I to the lipidated LC3B II, suggested also by the reduced LC3B I in all transduced cells compared to UTD cells. On the contrary, LC3B II/I ratio was not affected in WT hT cells.

Black asterisks refer to statistical comparison with the control cell line UTD (Friedman test followed by Dunn's test). Graphs show Mean with SEM.

FIG. 8. WB quantification of p62/WLN in all tested cell lines. A further autophagy marker is p62. The degradation of p62 level indicated the progression of autophagy flux. All transduced and WT hT cells had a lower amount of p62 protein, compared to UTD cells, indicating the enhancement of autophagy flux when ATM variants are present in AT cells, confirming the results obtained with the quantification of LC3B II/I analysis.

Black asterisks refer to statistical comparison with the control cell line UTD (Friedman test followed by Dunn's test). Graphs show Mean with SEM.

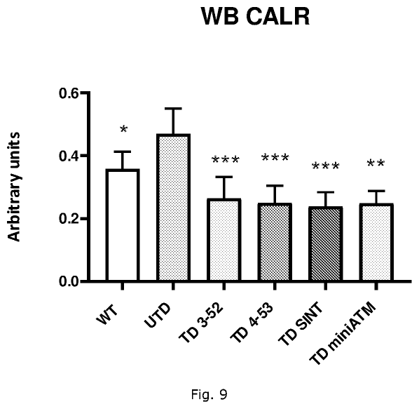

FIG. 9: WB quantification of CALR/WLN in all tested cell lines. Proteostatic stress was found in AT cells, with an enhanced expression of CALR under ER stress. The reduction of CALR amount in all ATM variants compared to UTD cells, could indicate an alleviation of ER stress when ATM variants are present.

Black asterisks refer to statistical comparison with the control cell line UTD (Friedman test followed by Dunn's test). Graphs show Mean with SEM.

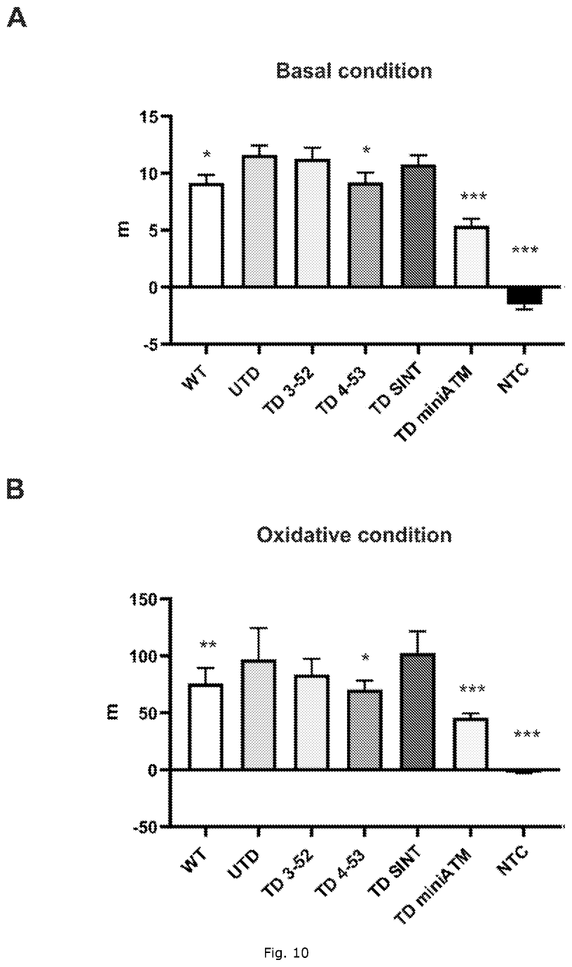

FIG. 10. ATM is activated after oxidative stress in the cytoplasm, in an independent manner from its activation after DNA damage. AT cells are in constant state of oxidative stress, and plasma of AT patients shows reduced antioxidant capacity. The Antioxidant capacity was tested in all the tested samples, evaluating the capacity of ATM variants to counteract the oxidation of the DCF probe under basal condition and after an oxidative stimulus (addition of H₂O₂). Only ATM 4-53 and miniATM counteracted the ROS production and had a greater antioxidant capacity than UTD cells, as happened in WT cells. Black asterisks refer to statistical comparison with the control cell line UTD (Kruskal Wallis followed by Dunn's test). Graphs show Mean with SEM.

FIG. 11. Mitochondria functionality was evaluated since ATM could be activated in response to mitochondria dysfunctions. Accordingly, ATM loss leads to a reduced mitochondria membrane potential and impaired mitophagy.

A: Mitochondrial membrane potential was evaluated staining all the tested samples with Mitotracker Red CMX-ROS, whose entry depends only on the mitochondria membrane potential of the cell line. All transduced and WT cells, expect for miniATM, demonstrated a better mitochondria functionality than UTD cells, which instead showed a leakage of the mitochondria potential. ATM 4-53 and ATM SINT showed a greater mitochondria membrane potential than ATM 3-52. Black asterisks refer to statistical comparison with the control cell line UTD (Welch's ANOVA test). Graphs show Mean with SEM. B: The accumulation of mtDNA in the cytoplasm indicated damaged mitochondria due to aberrant mitophagy. All transduced and WT cells had a lower accumulation of mtDNA in the cytoplasmic fraction than UTD cells, suggesting less damaged mitochondria or a greater clearance of depolarized mitochondria through mitophagy. ATM SINT showed the lowest mtDNA accumulation in the cytoplasm. Black asterisks refer to statistical comparison with the control cell line UTD. (Kruskal Wallis test followed by Dunn's test). Graphs show Mean with SEM. C: NAD+ amount was evaluated by mass spectrometry, since its depletion could be the cause of mitophagy impairment. All transduced and WT cells were able to recover its amount, with a greater extent in ATM SINT and miniATM, demonstrating their capacity in reverting AT mitophagy impairment. Black asterisks refer to statistical comparison with the control cell line UTD (Friedman test followed by Dunn's test).

FIG. 12. IF quantification of nuclear HDAC4 localization in all the tested samples. ATM is indirectly implicated in HDAC4 nuclear export, while ATM loss leads to HDAC4 nuclear import and neurodegeneration in AT neurons. All transduced cells were capable of altering HDAC4 nuclear/cytosol shuttle when are present in AT cells, showing a less accumulation of nuclear HDAC4. ATM SINT is the most efficient variant in reducing this accumulation, lowering it to the amount found in WT hT cells. Black asterisks refer to statistical comparison with the control cell line UTD (Kruskal-Wallis test followed by Dunn's test). Graphs show Mean with SEM.

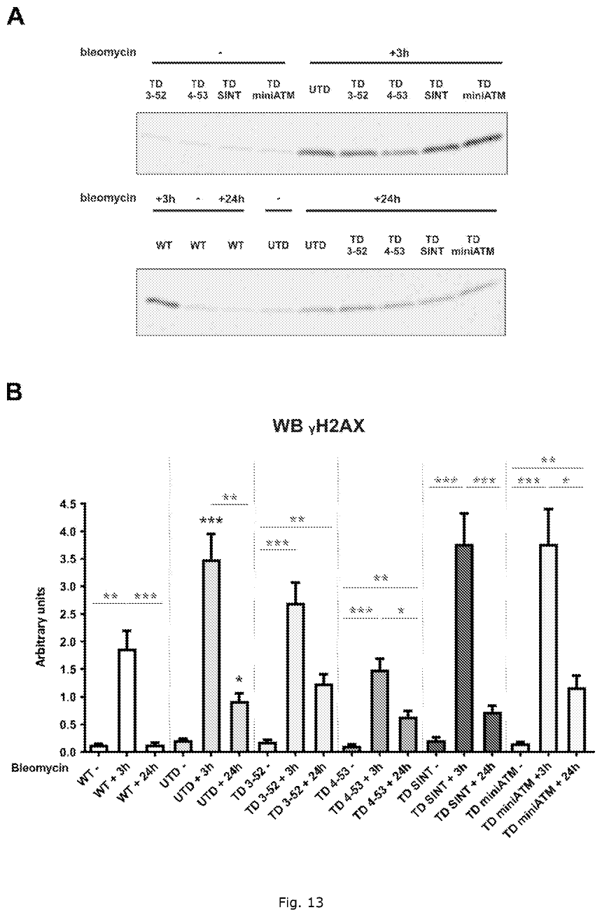

FIG. 13. Western blot quantification of yH2AX confirms IF analyses. A) Representative western blot and B) quantification of yH2AX of WT hT and AT 648 hT transduced and untransduced cells, treated with bleomycin for 3 h and kept in recovery for 24 hours. WT hT and AT 648 hT transduced cells were activated after bleomycin treatment and underwent a significant decrease after 24 h recovery. Same behavior was found in AT 648 hT untransduced cells. It has to be noted that Western Blot method does not allow to monitor individual nuclei analysis, that instead requires IF quantification (FIGS. 2 and 3). Red asterisks refer to intra sample comparison, while the black ones refer to statistical comparison with the control cell line UTD-(Friedman test followed by Dunn's test). Graphs show Mean with SEM.

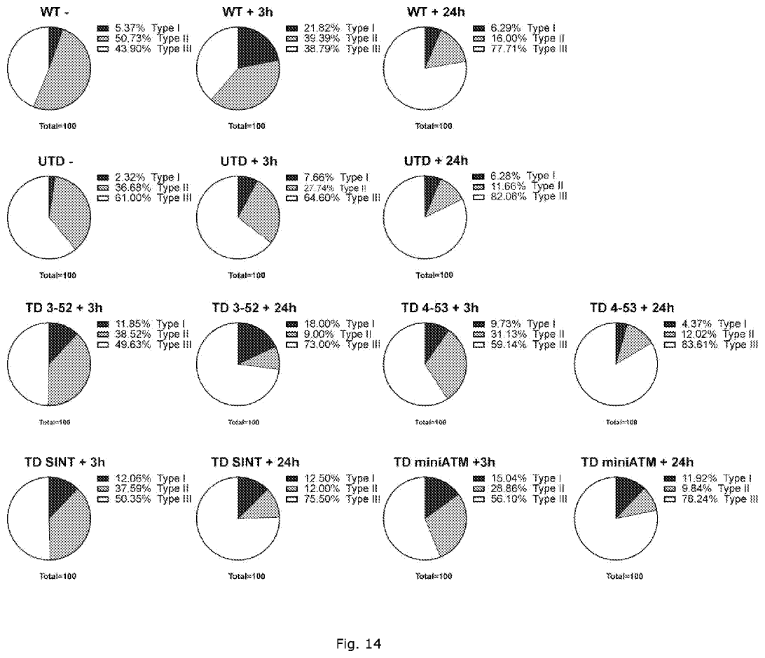

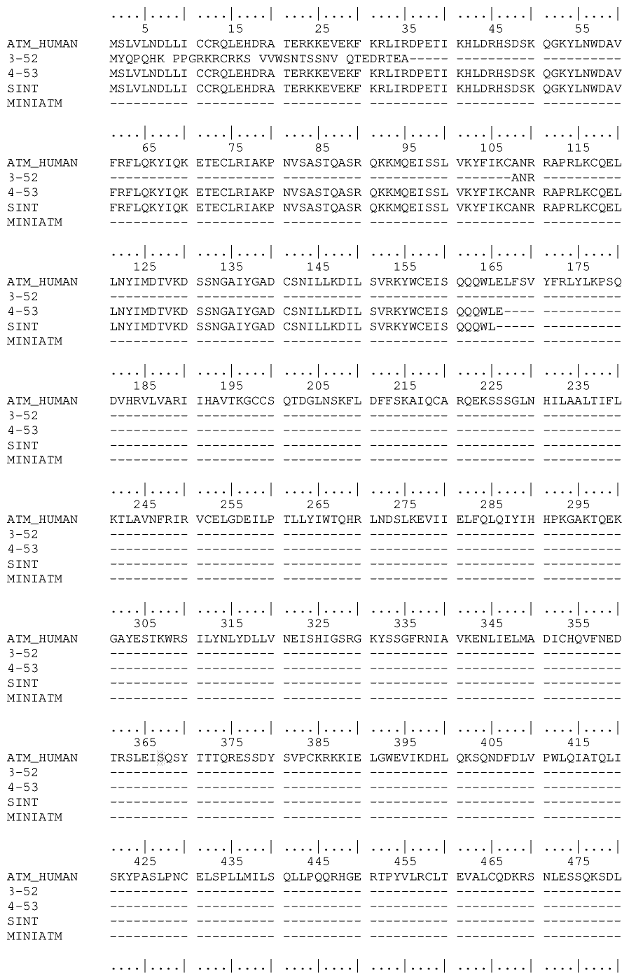

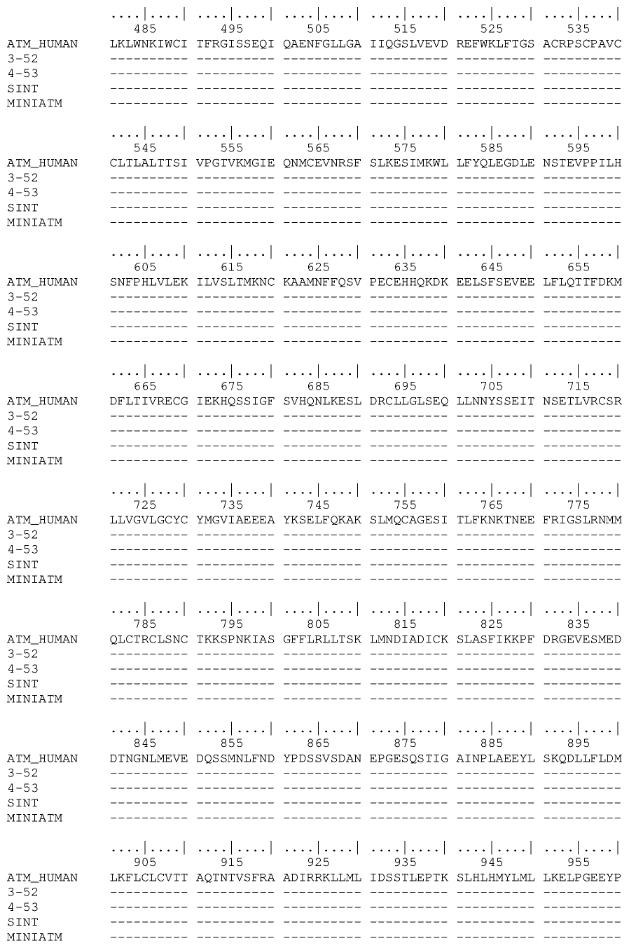

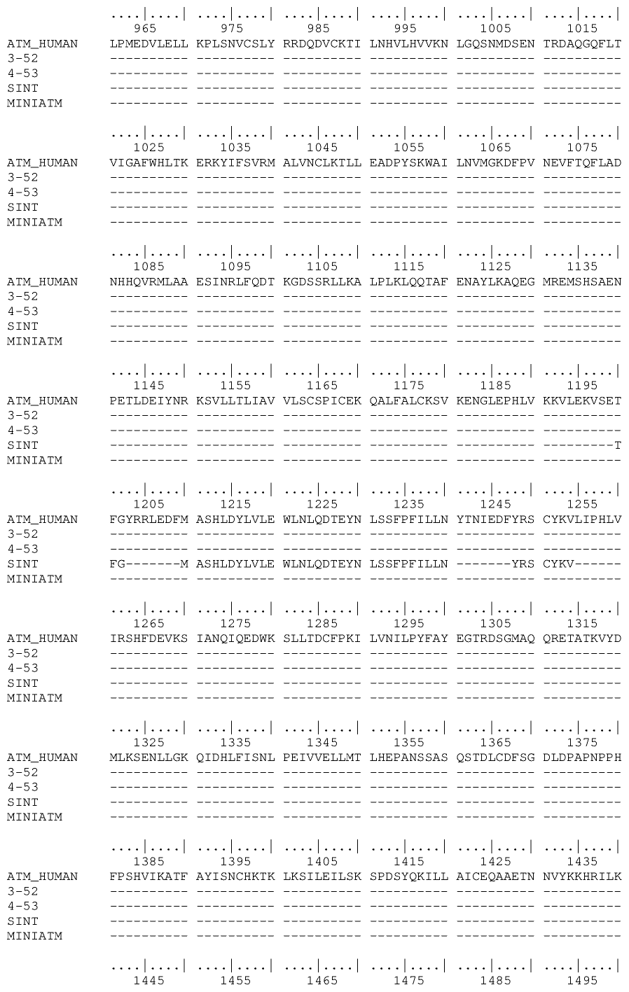

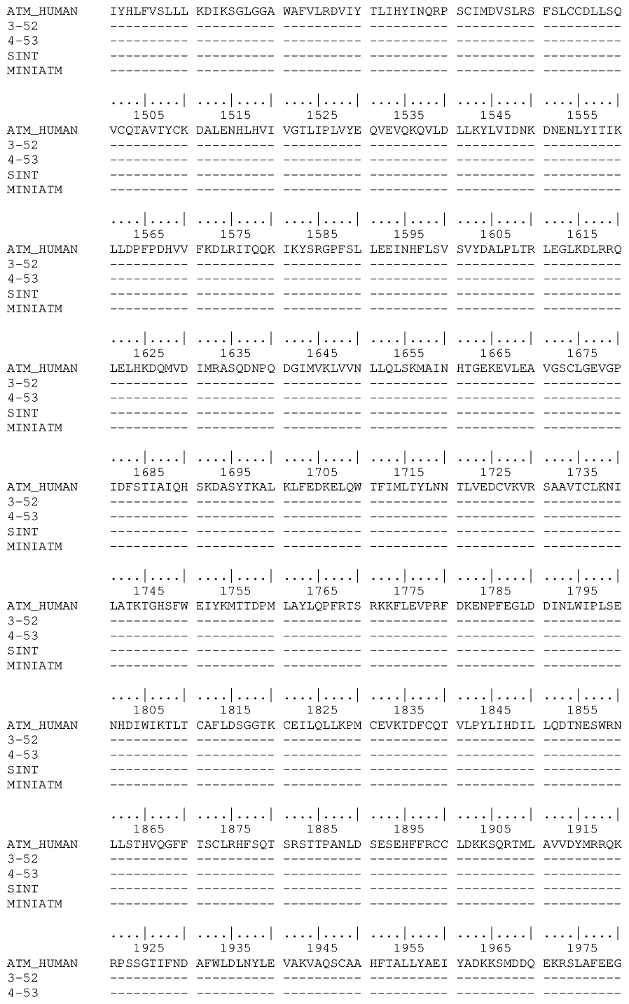

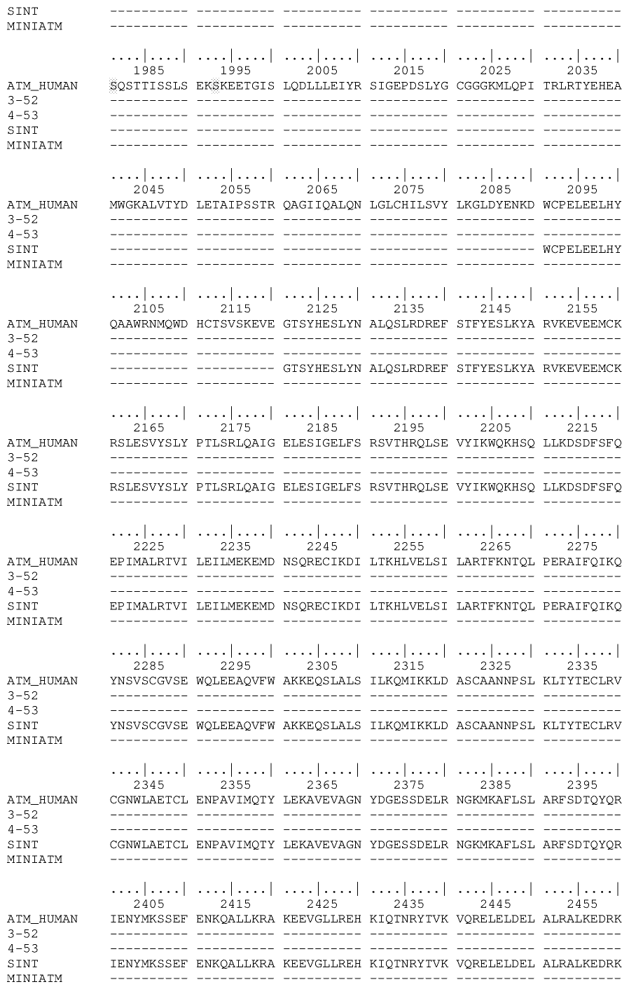

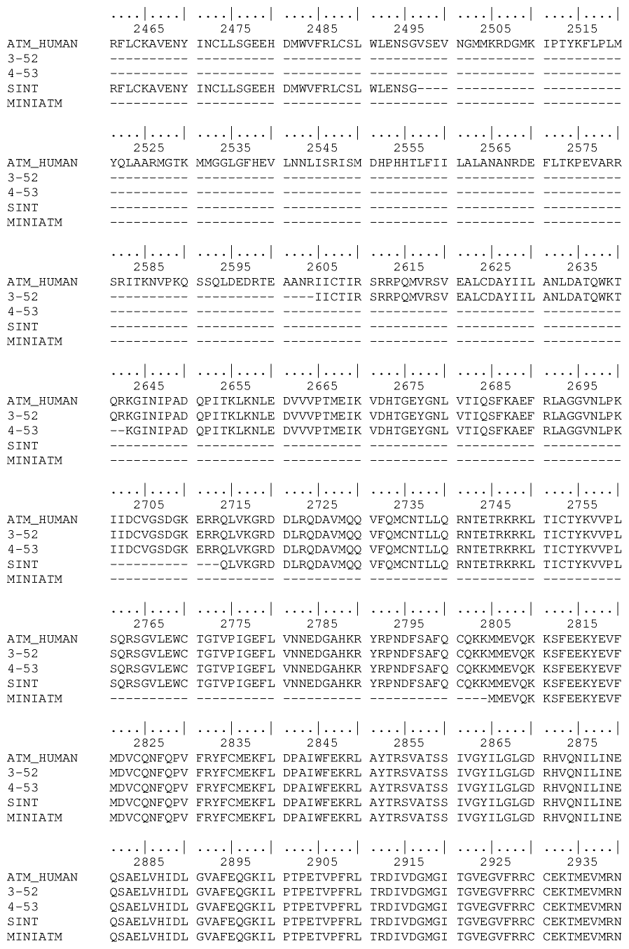

FIG. 14. Foci types staining of each tested cell line after each treatment condition. All ATM variants improved Type I stain pattern after 3 h of bleomycin treatment, suggesting an effective phosphorylation of H2AX in AT 648 hT transduced cells. In contrast, AT 648 hT untransduced cells have a slight increase of Type I foci over 3 h of drug, due to the lack of an active ATM. After 24 h of bleomycin, Type III staining type boosted in all the tested cell lines, despite different number of foci. (See Type III quantification in FIG. 3B).

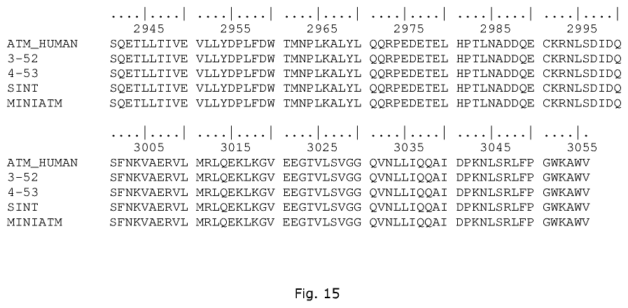

FIG. 15. Nucleotidic sequence alignment of ATM wild-type having SEQ ID NO 5, variant 3-52 having SEQ ID NO 2, variant 4-53 having SEQ ID NO 3, variant SINT having SEQ ID NO 1 and variant miniATM having SEQ ID NO 4.

SEQUENCES DESCRIPTION
SEQ ID NO 1-ATM SINT:
MSLVLNDLLICCRQLEHDRATERKKEVEKFKRLIRDPETIKHLDR
HSDSKQGKYLNWDAVFRFLQKYIQKETECLRIAKPNVSASTQASR
QKKMQEISSLVKYFIKCANRRAPRLKCQELLNYIMDTVKDSSNGA
IYGADCSNILLKDILSVRKYWCEISQQQWLTFGMASHLDYLVLEW
LNLQDTEYNLSSFPFILLNYRSCYKVWCPELEELHYGTSYHESLY
NALQSLRDREFSTFYESLKYARVKEVEEMCKRSLESVYSLYPTLS
RLQAIGELESIGELFSRSVTHRQLSEVYIKWQKHSQLLKDSDFSF
QEPIMALRTVILEILMEKEMDNSQRECIKDILTKHLVELSILART
FKNTQLPERAIFQIKQYNSVSCGVSEWQLEEAQVFWAKKEQSLAL
SILKQMIKKLDASCAANNPSLKLTYTECLRVCGNWLAETCLENPA
VIMQTYLEKAVEVAGNYDGESSDELRNGKMKAFLSLARFSDTQYQ
RIENYMKSSEFENKQALLKRAKEEVGLLREHKIQTNRYTVKVQRE
LELDELALRALKEDRKRFLCKAVENYINCLLSGEEHDMVVVFRLC
SLWLENSGQLVKGRDDLRQDAVMQQVFQMCNTLLQRNTETRKRKL
TICTYKVVPLSQRSGVLEWCTGTVPIGEFLVNNEDGAHKRYRPND
FSAFQCQKKMMEVQKKSFEEKYEVFMDVCQNFQPVFRYFCMEKFL
DPAIWFEKRLAYTRSVATSSIVGYILGLGDRHVQNILINEQSAEL
VHIDLGVAFEQGKILPTPETVPFRLTRDIVDGMGITGVEGVFRRC
CEKTMEVMRNSQETLLTIVEVLLYDPLFDVVTMNPLKALYLQQRP
EDETELHPTLNADDQECKRNLSDIDQSFNKVAERVLMRLQEKLKG
VEEGTVLSVGGQVNLLIQQAIDPKNLSRLFPGWKAWV*
SEQ ID NO 2-ATM 3-52:
MYQPQHKPPGRKRCRKSVVWSNTSSNVQTEDRTEAANRIICTIRS
RRPQMVRSVEALCDAYIILANLDATQWKTQRKGINIPADQPITKL
KNLEDVVVPTMEIKVDHTGEYGNLVTIQSFKAEFRLAGGVNLPKI
IDCVGSDGKERRQLVKGRDDLRQDAVMQQVFQMCNTLLQRNTETR
KRKLTICTYKVVPLSQRSGVLEWCTGTVPIGEFLVNNEDGAHKRY
RPNDFSAFQCQKKMMEVQKKSFEEKYEVFMDVCQNFQPVFRYFCM
EKFLDPAIWFEKRLAYTRSVATSSIVGYILGLGDRHVQNILINEQ
SAELVHIDLGVAFEQGKILPTPETVPFRLTRDIVDGMGITGVEGV
FRRCCEKTMEVMRNSQETLLTIVEVLLYDPLFDWTMNPLKALYLQ
QRPEDETELHPTLNADDQECKRNLSDIDQSFNKVAERVLMRLQEK
LKGVEEGTVLSVGGQVNLLIQQAIDPKNLSRLFPGWKAWV*
SEQ ID NO 3-ATM 4-53:
MSLVLNDLLICCRQLEHDRATERKKEVEKFKRLIRDPETIKHLDR
HSDSKQGKYLNWDAVFRFLQKYIQKETECLRIAKPNVSASTQASR
QKKMQEISSLVKYFIKCANRRAPRLKCQELLNYIMDTVKDSSNGA
IYGADCSNILLKDILSVRKYWCEISQQQWLEGINIPADQPITKLK
NLEDVVVPTMEIKVDHTGEYGNLVTIQSFKAEFRLAGGVNLPKII
DCVGSDGKERRQLVKGRDDLRQDAVMQQVFQMCNTLLQRNTETRK
RKLTICTYKVVPLSQRSGVLEWCTGTVPIGEFLVNNEDGAHKRYR
PNDFSAFQCQKKMMEVQKKSFEEKYEVFMDVCQNFQPVFRYFCME
KFLDPAIWFEKRLAYTRSVATSSIVGYILGLGDRHVQNILINEQS
AELVHIDLGVAFEQGKILPTPETVPFRLTRDIVDGMGITGVEGVF
RRCCEKTMEVMRNSQETLLTIVEVLLYDPLFDVVTMNPLKALYLQ
QRPEDETELHPTLNADDQECKRNLSDIDQSFNKVAERVLMRLQEK
LKGVEEGTVLSVGGQVNLLIQQAIDPKNLSRLFPGWKAWV*
SEQ ID NO 4-miniATM:
MMEVQKKSFEEKYEVFMDVCQNFQPVFRYFCMEKFLDPAIWFEKR
LAYTRSVATSSIVGYILGLGDRHVQNILINEQSAELVHIDLGVAF
EQGKILPTPETVPFRLTRDIVDGMGITGVEGVFRRCCEKTMEVMR
NSQETLLTIVEVLLYDPLFDWTMNPLKALYLQQRPEDETELHPTL
NADDQECKRNLSDIDQSFNKVAERVLMRLQEKLKGVEEGTVLSVG
GQVNLLIQQAIDPKNLSRLFPGWKAWV*
SEQ ID NO 5-ATM WILD-TYPE
MSLVLNDLLICCRQLEHDRATERKKEVEKFKRLIRDPETIKHLDR
HSDSKQGKYLNWDAVFRFLQKYIQKETECLRIAKPNVSASTQASR
QKKMQEISSLVKYFIKCANRRAPRLKCQELLNYIMDTVKDSSNGA
IYGADCSNILLKDILSVRKYWCEISQQQWLELFSVYFRLYLKPSQ
DVHRVLVARIIHAVTKGCCSQTDGLNSKFLDFFSKAIQCARQEKS
SSGLNHILAALTIFLKTLAVNFRIRVCELGDEILPTLLYIWTQHR
LNDSLKEVIIELFQLQIYIHHPKGAKTQEKGAYESTKWRSILYNL
YDLLVNEISHIGSRGKYSSGFRNIAVKENLIELMADICHQVFNED
TRSLEISQSYTTTQRESSDYSVPCKRKKIELGWEVIKDHLQKSQN
DFDLVPWLQIATQLISKYPASLPNCELSPLLMILSQLLPQQRHGE
RTPYVLRCLTEVALCQDKRSNLESSQKSDLLKLWNKIWCITFRGI
SSEQIQAENFGLLGAIIQGSLVEVDREFWKLFTGSACRPSCPAVC
CLTLALTTSIVPGTVKMGIEQNMCEVNRSFSLKESIMKWLLFYQL
EGDLENSTEVPPILHSNFPHLVLEKILVSLTMKNCKAAMNFFQSV
PECEHHQKDKEELSFSEVEELFLQTTFDKMDFLTIVRECGIEKHQ
SSIGFSVHQNLKESLDRCLLGLSEQLLNNYSSEITNSETLVRCSR
LLVGVLGCYCYMGVIAEEEAYKSELFQKAKSLMQCAGESITLFKN
KTNEEFRIGSLRNMMQLCTRCLSNCTKKSPNKIASGFFLRLLTSK
LMNDIADICKSLASFIKKPFDRGEVESMEDDINGNLMEVEDQSSM
NLFNDYPDSSVSDANEPGESQSTIGAINPLAEEYLSKQDLLFLDM
LKFLCLCVTTAQTNTVSFRAADIRRKLLMLIDSSTLEPTKSLHLH
MYLMLLKELPGEEYPLPMEDVLELLKPLSNVCSLYRRDQDVCKTI
LNHVLHVVKNLGQSNMDSENTRDAQGQFLTVIGAFWHLTKERKYI
FSVRMALVNCLKTLLEADPYSKWAILNVMGKDFPVNEVFTQFLAD
NHHQVRMLAAESINRLFQDTKGDSSRLLKALPLKLQQTAFENAYL
KAQEGMREMSHSAENPETLDEIYNRKSVLLTLIAVVLSCSPICEK
QALFALCKSVKENGLEPHLVKKVLEKVSETFGYRRLEDFMASHLD
YLVLEWLNLQDTEYNLSSFPFILLNYTNIEDFYRSCYKVLIPHLV
IRSHFDEVKSIANQIQEDWKSLLTDCFPKILVNILPYFAYEGTRD
SGMAQQRETATKVYDMLKSENLLGKQIDHLFISNLPEIVVELLMT
LHEPANSSASQSTDLCDFSGDLDPAPNPPHFPSHVIKATFAYISN
CHKTKLKSILEILSKSPDSYQKILLAICEQAAETNNVYKKHRILK
IYHLFVSLLLKDIKSGLGGAWAFVLRDVIYTLIHYINQRPSCIMD
VSLRSFSLCCDLLSQVCQTAVTYCKDALENHLHVIVGTLIPLVYE
QVEVQKQVLDLLKYLVIDNKDNENLYITIKLLDPFPDHVVFKDLR
ITQQKIKYSRGPFSLLEEINHFLSVSVYDALPLTRLEGLKDLRRQ
LELHKDQMVDIMRASQDNPQDGIMVKLVVNLLQLSKMAINHTGEK
EVLEAVGSCLGEVGPIDFSTIAIQHSKDASYTKALKLFEDKELQV
VTFIMLTYLNNTLVEDCVKVRSAAVTCLKNILATKTGHSFWEIYK
MTTDPMLAYLQPFRTSRKKFLEVPRFDKENPFEGLDDINLWIPLS
ENHDIWIKTLTCAFLDSGGTKCEILQLLKPMCEVKTDFCQTVLPY
LIHDILLQDTNESWRNLLSTHVQGFFTSCLRHFSQTSRSTTPANL
DSESEHFFRCCLDKKSQRTMLAVVDYMRRQKRPSSGTIFNDAFWL
DLNYLEVAKVAQSCAAHFTALLYAEIYADKKSMDDQEKRSLAFEE
GSQSTTISSLSEKSKEETGISLQDLLLEIYRSIGEPDSLYGCGGG
KMLQPITRLRTYEHEAMWGKALVTYDLETAIPSSTRQAGIIQALQ
NLGLCHILSVYLKGLDYENKDWCPELEELHYQAAWRNMQWDHCTS
VSKEVEGTSYHESLYNALQSLRDREFSTFYESLKYARVKEVEEMC
KRSLESVYSLYPTLSRLQAIGELESIGELFSRSVTHRQLSEVYIK
WQKHSQLLKDSDFSFQEPIMALRTVILEILMEKEMDNSQRECIKD
ILTKHLVELSILARTFKNTQLPERAIFQIKQYNSVSCGVSEWQLE
EAQVFWAKKEQSLALSILKQMIKKLDASCAANNPSLKLTYTECLR
VCGNWLAETCLENPAVIMQTYLEKAVEVAGNYDGESSDELRNGKM
KAFLSLARFSDTQYQRIENYMKSSEFENKQALLKRAKEEVGLLRE
HKIQTNRYTVKVQRELELDELALRALKEDRKRFLCKAVENYINCL
LSGEEHDMWVFRLCSLWLENSGVSEVNGMMKRDGMKIPTYKFLPL
MYQLAARMGTKMMGGLGFHEVLNNLISRISMDHPHHTLFIILALA
NANRDEFLTKPEVARRSRITKNVPKQSSQLDEDRTEAANRIICTI
RSRRPQMVRSVEALCDAYIILANLDATQWKTQRKGINIPADQPIT
KLKNLEDVVVPTMEIKVDHTGEYGNLVTIQSFKAEFRLAGGVNLP
KIIDCVGSDGKERRQLVKGRDDLRQDAVMQQVFQMCNTLLQRNTE
TRKRKLTICTYKVVPLSQRSGVLEWCTGTVPIGEFLVNNEDGAHK
RYRPNDFSAFQCQKKMMEVQKKSFEEKYEVFMDVCQNFQPVFRYF
CMEKFLDPAIWFEKRLAYTRSVATSSIVGYILGLGDRHVQNILIN
EQSAELVHIDLGVAFEQGKILPTPETVPFRLTRDIVDGMGITGVE
GVFRRCCEKTMEVMRNSQETLLTIVEVLLYDPLFDWTMNPLKALY
LQQRPEDETELHPTLNADDQECKRNLSDIDQSFNKVAERVLMRLQ
EKLKGVEEGTVLSVGGQVNLLIQQAIDPKNLSRLFPGWKAWV*
SEQ ID NO 6: Ser1981 phosphorylation domain
KSMDDQEKRSLAFEEGSQSTTISSLSEKSKEETGI
SEQ ID NO 7: Ser367 phosphorylation domain
ADICHQVFNEDTRSLEISQSYTTTQRESSDYSV
SEQ ID NO 8: Ser1893 phosphorylation domain
HFSQTSRSTTPANLDSESEHFFRCCLDKKSQRTMLA
SEQ ID NO 9: Ser794 phosphorylation domain
LCTRCLSNCTKKSPNKIASGFFLRLLTSKLM
SEQ ID NO 10: Ser1403 phosphorylation domain
AYISNCHKTKLKSILEILSKLKSILEILSKSP

### DETAILED DESCRIPTION OF THE INVENTION

The Authors of the present invention have found that the transduction of fibroblast cells with ATM variants of reduced size (e.g. variants of a size of less than about 1000 amino acids) with respect to the full length ATM through a lentiviral system, achieved a high transduction efficiency (almost 100% of the cells resulted transduced) due to their reduced cDNA size, improving the efficiency of viral particles production and infection efficiency than whole ATM gene, and that the usage of ATM variants of said size is capable of overcoming the cargo limit of the actual vectors approved for gene therapy to treat AT patients.

In particular, as disclosed above, the authors of the present invention have designed an artificial variant of human ATM of a size lower than 1000 amino acids, which is capable of effectively restoring a number of human ATM functions and which is, overall, more effective than the miniATM variant known in the art.

The present invention therefore relates to a variant of the human ATM protein having SEQ ID NO 1, or a derivative thereof.

It is understood that the mere substitution of one or more amino acid of SEQ ID NO 1 with one or more functionally equivalent amino acid (i.e. leucine might be replaced by another non-polar amino acid such as isoleucine), thereby resulting in a protein that is (as expected) equivalent in function and structure to the variant of the human ATM protein coded by SEQ ID NO 1, is considered an equivalent of SEQ ID NO 1.

In other words, the invention relates to a variant of the human ATM protein designed in silico, herein also named ATM SINT, which is capable of restoring most of the functions of the ATM wild type such as: DNA DSBs repair, autophagy progression, activation of mitochondria functionality and capacity to alter HDAC4 localization promoting HDAC4 nuclear export, and that at the same time overcomes the cargo limit of vectors presently approved for gene therapy.

The derivative of the variant of the human ATM protein of SEQ ID NO 1 according to the invention, is described in the embodiments below and is functionally characterised by retaining at least all the restoring capabilities of the variant of SEQ ID NO 1, i.e., DNA DSBs repair, autophagy progression, activation of mitochondria functionality and capacity to alter HDAC4 localization promoting HDAC4 nuclear export.

Structurally, in an embodiment of the invention, said derivative is characterized in that it further comprises from 3 to 170, preferably 3 to 135, or 3 to 110, more preferably 3 to 80, even more preferably 3 to 65 and further more preferably 3 to 40 additional amino acids between the amino acids in position 205 and 206 of SEQ ID NO 1. Amino acids in positions 205 and 206 of SEQ ID NO 1 are, respectively, V (Valine) and W (Tryptophan), therefore said derivative comprises further amino acids between V in position 205 and W in position 206 of SEQ ID NO 1.

In a preferred embodiment of the invention, said 3 to 170, preferably 3 to 135, or 3 to 110, more preferably 3 to 80, even more preferably 3 to 65 and furthermore preferably 3 to 40 additional amino acids code for one or more phosphorylation domain selected from the human ATM protein having SEQ ID NO 5 (wild type).

In an embodiment of the invention, said additional amino acids comprise one or more phosphorylation site of wild type human ATM having SEQ ID NO 5.

Phosphorylation site, in the present description, is intended as an amino acid that is naturally subject to phosphorylation in wild type human ATM having SEQ ID NO 5. In the present description, the expression phosphorylation domain defines a portion of a protein (in the present case the wild-type human ATM protein of SEQ ID NO 5), and contiguous amino acids N′ and C′ adjacent to said serine, i.e. a fragment of 15 to 40, preferably 20 to 36, even more preferably 30 to 36, contiguous amino acids of SEQ ID NO 5 comprising one or more amino acid naturally phosphorylated (preferably a serine).

In a preferred embodiment of the invention, said one or more phosphorylation domain is a domain comprising Serine (S) in position 1981 of SEQ ID NO 5, or a domain comprising Serine (S) in position 1893 of SEQ ID NO 5 or a domain comprising Serine (S) in position 367 of SEQ ID NO 5, or a domain comprising Serine (S) in position 794 of SEQ ID NO 5, or a domain comprising Serine (S) in position 1403 of SEQ ID NO 5.

By way of example, suitable phosphorylation domains can be selected from: a domain comprising Serine (S) 1981 of SEQ ID NO 6, a domain comprising Serine (S) 367 of SEQ ID NO 7, a domain comprising Serine (s) 1893 of SEQ ID NO 8, a domain comprising Serine (S) of 794 SEQ ID NO 9, and a domain comprising Serine (S) 1403 of SEQ ID NO 10.

In a preferred embodiment, the derivative of the variant of the human ATM protein having SEQ ID NO 1 comprises one or two of the phosphorylation domains defined above.

The invention also relates to the variant of the human ATM protein having SEQ ID NO 1, or a derivative thereof, according to any one of the embodiments herein disclosed, for use as a medicament.

In particular, the invention relates to the variant of the human ATM protein having SEQ ID NO 1, or a derivative thereof according to any one of the embodiments herein disclosed for use in the prevention, or in the treatment, or in adjuvating the treatment of a disease related to at least one mutation of the ATM gene/s. Said mutations can be either monoallelic or biallelic (i.e. affecting one or both ATM genes).

In particular, said mutations can be in the germline cells and/or in the somatic cells. Examples of said diseases are Ataxia Telangiectasia and cancer. A non-limiting example of said cancer comprises mantle cell lymphoma, T-cell prolymphocytic leukemia, Cutaneous squamous cell carcinoma, Hepatocellular, Colorectal, Neuroendocrine prostate, Diffuse large B-cell lymphoma, Uterine endometrioid carcinoma, Bladder urothelial, Prostate adenocarcinoma, Stomach adenocarcinoma, Lung adenocarcinoma, Primary CNS lymphoma, Cervical squamous or adenocarcinoma, Uterine carcinosarcoma, Chronic lymphocytic leukaemia, Anaplastic thyroid cancers, Melanoma, Small cell lung cancer, Malignant peripheral nerve sheath tumour, Lung squamous cell carcinoma, Pancreatic adenocarcinoma, Head and neck squamous cell carcinoma, Testicular germ cell cancer, Breast cancer, Breast invasive carcinoma, Paediatric Ewing sarcoma, Kidney renal clear cell carcinoma, Gallbladder, Glioblastoma, Ovarian serous cystadenocarcinoma, Multiple myeloma, Oesophageal adenocarcinoma, Adenoid cystic carcinoma, Acute myeloid leukaemia, Oesophageal squamous cell carcinoma, Papillary thyroid carcinoma, Medulloblastoma, which are also reported in Table 1 and FIG. 3 of Michael Choi et al. ATM Mutations in Cancer: Therapeutic Implications. Molecular Cancer Therapeutics. 2016 Jul. 13; DOI: 10.1158/1535-7163.MCT-15-0945.

In a preferred embodiment of the invention, said cancer form, in which the affected subject has a monoallelic mutation, i.e., a single mutated AMT gene, is breast cancer. The invention encompasses cancer prevention as it is well-known in the art that patients with certain ATM biallelic mutations are predicted to develop cancer and show a significantly reduced life expectancy due to cancer, in particular female patients having particular ATM alleles have a statistically assessed increased risk of developing breast cancer.

Cancer development in subjects carrying ATM mutations is expected due to the loss of various functions of the ATM protein, in particular DSB repairs. By way of example, Choi et al, 2016, reports studies of family members known to be biallelic for ATM gene mutations showed an approximate 2- to 3-fold risk of cancer, and a 5- to 9-fold risk of breast cancer in women. Hence, the state of the art teaches that impaired ATM function caused by mutation/s of the ATM gene, appears to be crucial for the development of various cancers.

Therefore, it can be directly derived from the state of the art that, by restoring ATM functions, the variant of the human ATM protein having SEQ ID NO 1, and/or a derivative thereof as herein defined, are suitable for use in the prevention of cancer onset, in particular breast cancer, in the specific cohort of patients carrying ATM biallelic gene mutations.

Also, a large number of monoallelic mutations are well known in the art for being directly related to cancer development, being at least a co-cause of the onset and of the severity of the disease.

The malfunction or total loss of function of the ATM protein coded by the mutated ATM gene/s, either in monoallelic or biallelic form is hence known to be a direct cause or a co-cause of all the above-mentioned diseases in subjects carrying said mutation/s. Therefore, the variant of the human ATM protein and/or derivatives thereof of the present invention can advantageously be used in the prevention, in the treatment or in adjuvating the treatment of a disease related to at least one mutation of the ATM gene/s, said mutations being either monoallelic or biallelic (one or both genes), in particular, in adjuvating the treatment of cancer in patients carrying said mutation/s in which the mutation/s cause the malfunction or total loss of function of the ATM protein coded by the mutated ATM gene/s.

According to the invention said mutations can be in the germline cells and/or in the somatic cells.

In addition, the cohort of patients carrying one or more mutation in the ATM gene/s, in which the mutation/s cause the malfunction or total loss of function of the ATM protein coded by said mutated ATM gene/s have been reported to have particularly severe adverse effects upon treatment with various accepted cancer treatment protocols.

A non-limiting example of cancer treatment that can be advantageously adjuvated by the variant and/or derivatives thereof as herein defined in said cohort of patients, comprises radiotherapy, chemotherapy, treatment with platinum drugs, treatment with PARP inhibitors, treatment targeting ATR, treatment with CHK1 inhibitors. As also reported by Choi et al. 2016, increased toxicity to radiotherapy has been reported in patients with A-T syndrome and biallelic carriers of ATM mutations, probably due to defective DNA repair and genomic instability in normal tissues. Similarly, toxicity from systemic chemotherapy may be increased in patients with biallelic germ-line ATM variants subjected to chemotherapy.

Similarly, monoallelic mutations of the ATM gene causing malfunction or loss of functions appear to cause increased toxicity of various cancer treatments. The variant of the human ATM protein of the present invention and/or derivatives thereof as herein defined, may hence be used in adjuvating said treatments thanks to the fact that it possesses the function of restoring at least in part the repair of DNA DSBs and other functions (see Table 1) of the wild-type human ATM protein.

Therefore, by restoring a number of ATM functions, the variant of the human ATM protein having SEQ ID NO 1, and/or a derivative thereof as herein defined is suitable for use in adjuvating the treatment of a disease related monoallelic or biallelic to mutation/s of the ATM gene, wherein said mutations can be in the germline cells and/or in the somatic cells.

Therefore, the invention encompasses the combination of the variant having SEQ ID NO 1 and/or a derivative thereof as herein defined, with, a drug cocktail or with one or more drug, therapeutically active principle or drug as well as the association therapeutic regimen for the treatment of cancer and the administration of the ATM variant having SEQ ID NO 1 and/or derivatives thereof as herein defined.

In all the medical use embodiments of the invention the variant and/or derivative thereof can be administered in the form of a polypeptide optionally combined with suitable carriers or complexed with suitable molecules, or in the form of a nucleotide or a nucleotide delivery system as described herein.

In an embodiment, the variant and/or derivative thereof is expressed by the host following administration of a suitable gene therapy nucleotide construct.

In an embodiment of the invention, the variant of the human ATM protein having SEQ ID NO 1, or a derivative thereof, can be co-administered with at least one variant of the human ATM protein having SEQ ID NO 2, SEQ ID NO 3 or SEQ ID NO 4.

Also, for this embodiment the combination with a drug cocktail or with at least one additional therapeutically active compound or drug or, alternatively, the association with chemical, radiological or immunological cancer therapy is encompassed by the present invention.

Fusion proteins comprising the variant of the invention and one or more derivative thereof and/or one or more additional variant of seq id 2-4 and their uses in any of the embodiments herein described for the variant of SEQ ID NO 1 are also encompassed by the present invention.

A further object of the present invention is a nucleotide sequence coding for the variant of the human ATM protein having SEQ ID NO 1, or a derivative thereof as defined in the previously described embodiments and in the claims.

According to the invention, said nucleotide sequence can advantageously comprise optimised codons, selected in order to match with the more abundant tRNAs of the organism to which said mRNA will be administered. In a preferred embodiment the codons can be optimised for humans.

In an embodiment of the invention, said nucleotide sequence is a cDNA or a mRNA.

In a preferred embodiment of the invention, said nucleotide sequence is a mRNA. Preferably, said mRNA comprises a 3′ and a 5′ UTR element flanking the coding sequence, a 5′ Cap and a polyA tail. 5′ and 3′ mRNA untranslated regions (UTR) are well known in molecular genetics. 5′ UTR is a sequence containing a Kozak consensus sequence that is recognised by the ribosome and allows the ribosome to bind to the mRNA molecule and to initiate its translation. 3′ UTR region is found after the stop codon and has a role in translation termination and post-transcriptional modifications. UTRs are well known to the skilled person as well as is their role for enhanced protein production in non-viral gene therapies. The skilled person can readily select among UTRs commonly used in non-viral gene therapy a suitable 5′ and 3′ sequence for the mRNA construct of the present invention.

Additionally, said mRNA may also comprise a 5′ cap and a polyA tail. Both stabilise the mRNA molecule and increase protein translation. Various versions of 5′ caps known in the art can be added during or after the transcription reaction using a vaccinia virus capping enzyme or by incorporating synthetic cap or anti-reverse cap analogues.

In an embodiment of the present invention, the mRNA molecule, in any of the embodiments herein disclosed, can comprise one or more modified nucleosides in order to enhance the stability, the safety and/or the translation of the same. Suitable examples of modified nucleosides include but are not limited to pseudouridine and 1-methylpseudouridine.

Still in another embodiment, that applies to all the embodiments related to the mRNA molecule of the invention, said molecule can be naked or can be complexed with one or more carrier molecules according to the common knowledge in the art in the form, by way of example, of a in a cationic nanoemulsion, nanoparticle, liposome, cationic polymer liposome, polysaccharide particle cationic lipid nanoparticle, cationic lipid cholesterol nanoparticle or cationic lipid cholesterol PEG nanoparticle.

Commonly used delivery methods and carrier molecules for therapeutic mRNA molecules include: naked mRNA (part a); naked mRNA with in vivo electroporation; protamine (cationic peptide)-complexed mRNA; mRNA associated with a positively charged oil-in-water cationic nanoemulsion; mRNA associated with a chemically modified dendrimer and complexed with polyethylene glycol (PEG)-lipid; protamine-complexed mRNA in a PEG-lipid nanoparticle; mRNA associated with a cationic polymer such as polyethylenimine (PEI); mRNA complexed with a cationic polymer such as PEI and a lipid component; mRNA complexed with a polysaccharide (for example, chitosan) particle or gel; mRNA in a cationic lipid nanoparticle (for example, 1,2-dioleoyloxy-3-trimethylammoniumpropane (DOTAP) or dioleoylphosphatidylethanolamine (DOPE) lipids); mRNA complexed with cationic lipids and cholesterol (part k); and mRNA complexed with cationic lipids, cholesterol and PEG-lipid.

The cationic peptide protamine has been shown to protect mRNA from degradation by serum RNases.

In an embodiment of the invention, said nucleotide sequence is for use as a medicament.

In a preferred embodiment of the invention, said nucleotide sequence is for use in the prevention or in the treatment or in adjuvating the treatment of a disease related to at least one mutation of the ATM gene/s. Said mutation can be monoallelic or biallelic (one or both genes). In particular, said mutations can be in the germline cells and/or in the somatic cells.

Examples of said diseases that comprise Ataxia Telangiectasia and cancer are provided above.

In a preferred embodiment of the invention, said cancer form in which the affected subject has a monoallelic mutation, i.e. a single mutated AMT gene, is breast cancer. All the embodiments described above with respect to for the variant of SEQ ID NO 1 and/or derivatives thereof on the medical use/treatment apply, mutatis mutandis, to nucleotide sequences coding for the variant of SEQ ID NO 1 and/or derivatives thereof.

Another object of the invention is an expression vector, comprising a nucleotide sequence coding for the variant of the human ATM protein having SEQ ID NO 1, or a derivative thereof as defined in the previous embodiments, operably linked to a promoter. The promoter may be a constitutive promoter, an inducible promoter, a ubiquitous promoter, a tissue specific promoter, preferably a constitutive promoter, thereby providing the expression of the desired therapeutic protein.

In an embodiment of the invention, said nucleotide sequence is a cDNA or a RNA comprised in said expression vector is a cDNA or a RNA.

In an embodiment of the invention, said expression vector is selected from a plasmid, a yeast vector, a mammalian vector, a viral vector, a gene therapy expression vector, a single-stranded phage, a double-stranded phage, artificial chromosome.

Preferably, said gene therapy expression vector is selected from the following list: adenovirus, adeno-associated virus (AAV), lentivirus, retrovirus, cytomegalovirus (CMV) Herpes Simplex Virus (HSV). In a preferred embodiment the cDNA of the invention may be designed in order to contain optimised codons.

In an embodiment of the invention, said variant of the human ATM protein or a derivative thereof is recombinantly expressed by a lentiviral vector.

Therefore, another embodiment of the present invention relates to a gene therapy expression lentiviral vector, comprising a DNA sequence (cDNA) coding for the variant of the human ATM protein having SEQ ID NO 1, or a derivative thereof.

Preferably, said vector comprising said cDNA will be designed in order to provide the recombinant expression of a variant of the same species to be treated, therefore, in a preferred embodiment, when the subject is a human subject, the cDNA of the human gene will be expressed resulting in the expression of the recombinant human variant of interest, or a derivative thereof.

In an embodiment of the invention, said gene therapy expression vector is for use as a medicament.

Preferably, said expression vector is for use in the prevention or in the treatment or in adjuvating the treatment of a disease related to at least one mutation of the ATM gene/s; said mutation being either monoallelic or biallelic (one or both genes). In particular, said mutations can be in the germline cells and/or in the somatic cells.

Examples of said diseases that comprise Ataxia Telangiectasia and cancer are provided above.

In a preferred embodiment of the invention, said cancer form in which the affected subject has a monoallelic mutation, i.e., a single mutated AMT gene, is breast cancer.

According to any one of the embodiments of the present invention, said gene expression vector coding for the variant of the human ATM protein having SEQ ID NO 1, or a derivative thereof, is administered in a therapeutically effective amount to a patient in need thereof, optionally in combination with at least one variant of the human ATM protein having SEQ ID NO 2, SEQ ID NO 3 or SEQ ID NO 4 and/or a drug cocktail or at least one additional therapeutically active compound or drug, or in association to a chemical, radiological or immunological cancer therapy. In order to overcome the cargo limit of the actual vectors approved for gene therapy, said combination can be administered as a chimera of the selected variants, or the variant can be administered in “trans”, that is, simultaneously through two different vectors, or in a vector coding for a fusion of one or more of the variants mentioned above.

Both the mRNA or the expression vector of the invention can be administered in combination with a further pharmaceutically active compound or drug or drug cocktail or in addition to a cancer therapy.

A further object of the present invention is a pharmaceutical composition comprising the variant of the human ATM protein or a derivative thereof according to any one the embodiments herein disclosed in the form of a recombinant protein, or the mRNA according to any one the embodiments herein disclosed, or the expression vector according one the embodiments herein disclosed; and a pharmaceutically acceptable carrier and/or excipient. When mRNA is used, the molecule can be formulated with a transfection reagent. Suitable transfection reagents are commercially available. Suitable pharmaceutical carriers and excipients are well-known to the skilled person.

In an embodiment of the invention, said pharmaceutical composition may be further comprising one or more variant of the human ATM protein having SEQ ID NO 2, 3 or 4, or the mRNA or a nucleotide sequence or an expression vector coding for said one or more variant.

Furthermore, in an embodiment of the invention according to any one of the embodiments herein disclosed, said pharmaceutical composition is for use as a medicament.

Still in a further embodiment of the invention, said pharmaceutical composition is for use in the prevention or in the treatment or in adjuvating the treatment of a disease related to at least one mutation of the ATM gene/s (one or both genes); said mutation can be either monoallelic or biallelic. In particular, said at least one mutation can be in the germline cells and/or in the somatic cells.

Examples of said diseases that comprise Ataxia Telangiectasia and cancer are provided above.

In a preferred embodiment of the invention, said cancer form in which the affected subject has a monoallelic mutation, i.e., a single mutated AMT gene, is breast cancer.

The pharmaceutical composition of the present invention is preferably a composition suitable for systemic injection, central nervous system delivery, aerosol/nasal delivery, topical delivery RBCs and vesicles delivery, especially for intravenous injection administration, intraparenchymal administration in particular areas of the brain such as intracerebroventricular, cisternal, lumbar or intrathecal administration, or intra-arterial injection administration, or for direct administration into the cerebrospinal fluid.

The pharmaceutical composition of the invention can be administered in combination with a drug cocktail or at least one additional therapeutically active compound or drug or in combination with a chemical, radiological or immunological cancer therapy.

A further object of the invention is a combination of said variant having SEQ ID NO 1 or derivative thereof or of said mRNA, nucleotide sequence or expression vector coding for the variant of the human ATM protein having SEQ ID NO 1, or a derivative thereof, or of said pharmaceutical composition and at least one additional therapeutically active compound or drug, a drug cocktail.

An additional object of the invention is the variant having SEQ ID NO 1 or derivative thereof or said mRNA, nucleotide sequence or expression vector coding for the variant of the human ATM protein having SEQ ID NO 1, or a derivative thereof, or said pharmaceutical composition a chemical in association to a radiological, or an immunological cancer therapy for use in the treatment or in adjuvating the treatment of a disease related to at least one mutation of the ATM gene/s, wherein said mutation can be either monoallelic or biallelic; the disease being as defined in the present description.

The invention also encompasses a method for the prevention or the treatment or for adjuvating the treatment of a disease related to at least one mutation of the ATM gene/s. According to the invention said mutation can be either monoallelic or biallelic (one or both genes). In particular, said mutations can be in the germline cells and/or in the somatic cells. According to the methods indicated above, a therapeutic effective amount of a variant, a nucleotide molecule, a vector, a pharmaceutical composition according to any of the embodiments herein disclosed or claimed is administered in one or more dosages to a patient in need thereof.

The method also encompasses the co-administration of therapeutic effective amount of a variant, a nucleotide molecule, a vector, a pharmaceutical composition according to any of the embodiments herein disclosed or claimed in combination with at least one additional therapeutically active compound or drug, a drug cocktail or in association with a chemical, a radiological, or an immunological cancer therapy In a preferred embodiment, the “therapeutically active compound” according to the present description is a compound which is used in the art for the treatment of a disease related to at least one mutation of the ATM gene/s, i.e., a disease caused or co-caused by said at least one mutation.

Examples of said diseases that comprise Ataxia Telangiectasia and cancer are provided above.

In a preferred embodiment of the invention, said cancer form in which the affected subject has a monoallelic mutation, i.e. a single mutated AMT gene, is breast cancer. Examples are reported below which have the purpose of better illustrating the methodologies disclosed in the present description, such examples are in no way to be considered as a limitation of the previous description and the subsequent claims. In compliance with art. 170bis of the Italian patent law it is declared that all the experiments concerning the cells were carried out on cells available on the market.

### EXAMPLES

### Materials and Methods

### Cell Culture and Treatments

Fibroblasts WT AG09429 (Atm^{+/+}) and AT GM00648 (Atm^{-/-}) from Coriell Institute (Camden, NJ, USA) were used as a cellular model. The hTERT antigen cell immortalization Kit (Alstem Cell Advancements) was used to immortalize the cells. The selected AT GM00648 hTERT (AT 648 hT) and WT AG09429 hTERT (WT ht) were grown in MEM (Eagle formulation). The medium was supplemented with 2 mmol/L L-glutamine, 100 U/mL penicillin, and 0.1 mg/mL streptomycin (Sigma Aldrich), 15% fetal bovine serum (Thermo Fisher Scientific) and 10 mM glucose. All cells were incubated at 37° C. with 5% CO2. Human embryonic kidney (HEK) 293T cells (ATCC® CRL-3216™), used for transfection in lentiviral particles production, were grown in D-MEM (Eagle formulation). The medium was supplemented with 2 mmol/L L-glutamine, 100 U/mL penicillin, and 0.1 mg/mL streptomycin (Sigma Aldrich), and 10% fetal bovine serum (MERK).

### ATM Variants Description

ATM 3-52 possesses the complete Phosphatidyl Inositol 3 Kinase (P13K) and the FAT-C-terminal (FATC) domains (miniATM contains only a partial P13K) because its translation could start 714 bp upstream than miniATM starting codon, even though not the native one. ATM 4-53 (1740 bp) splicing derived ATM messenger can be translated from the native starting codon. This confers to the translated protein the Telomere-length maintenance and DNA damage repair (TAN) domain as well as the full P13K and FATC domains. A third variant named ATM SINT has been designed in silico and it contains the native starting codon with the TAN domain, the leucine zipper domain, the FAT domain (FRAP-ATM-TRRAP: from amino acids 2123 to 2496, purposely achieved without Ser1981), the P13K and FATC domains.

The sequences of the tested variants are reported in the detailed description of the sequences and in the annexed sequence listing.

### Lentiviral Vector Construction and Production

ATM 3-52, ATM 4-53, ATM SINT and miniATM cDNAs were inserted into pLenti-C-Myc-DDK-IRES-Neo Tagged Cloning Vector with double selection: Chloramphenicol for E. coli selection and Neomycin for mammalian cell selection. WT hT and AT 648 hT not transduced cells were used as reference and negative control, respectively. Viral particles were produced by co-transfecting HEK 293T cells in 24-well plates (1.2×105/well) with cloned ATM variants using MegaTran1.0 Transfection Reagent as reported by the Lenti-vpak Lentiviral Packaging Kit (OriGene). Viral particles were collected and concentrated according to the method reported by Miller et al., 1996 (A rapid and efficient method for concentration of small volumes of retroviral supernatant. Nucleic acids research 1996, 24(8):1576-1577.) on Pag. 1576, second paragraph in the second column.

### Transduction of Cells

4×104 AT 648 hT cells per well were seeded in 24-well plates and after 24 h, viral particles were added to cells in the presence of 5 μg/ml of polybrene (MERK). Clones' selection was performed as indicated by the Lenti-vpak Lentiviral Packaging Kit supplier. AT 648 hT transduced cell lines were called TD 3-52 for ATM 3-52, TD 4-53 for ATM 4-53, TD SINT for ATM SINT and TD miniATM for miniATM. UTD is referred to AT 648 hT untransduced cells.

### DSBs Induction by Bleomycin

To determine whether the ATM constructs were efficient to counteract the DNA damage response, WT hT and AT 648 hT transduced and untransduced cells were treated with bleomycin at a final concentration of 8 μg/ml. Cells underwent three types of treatment: placebo solution, 3 h of bleomycin treatment, and subsequent 24 h incubation in drug-free culture medium.

### Western Blotting

Total proteins were extracted using the Protein Extraction Reagent Type 4 (P4, Sigma Aldrich). Cells were sonicated with 10 pulses of 15 seconds at 45 Watts Labsonic 1510 Sonicator (Braun) and clarified by centrifugation for 10 minutes at 10 000 RCF. Protein concentration was determined by the Bio-Rad Protein Assay, based on Bradford's method. Twenty micrograms of proteins were separated by SDS-PAGE (Novex TrisGlycine gels) according to the Laemmli protocol [3] and then transferred to nitrocellulose (0.22 μm, Bio-Rad) or LF PVDF (0.45 μm, Bio-Rad) by wet transfer and Towbin blotting buffer (50 mM Tris, 150 mM NaCl, 20% v/v methanol). Membranes were probed with the primary antibodies diluted in 5% w/v non-fat dry milk or 5% BSA in TBS-T. The primary antibodies used in this study were: anti-phospho H2AX Ser139 (GeneTex and Cell Signaling Technology, CST), anti-phospho p53 Ser15 (CST), anti-p53 (Santa Cruz Biotechnology, SCBT), anti-phospho CHK2 Thr68 (CST and AB clonal), anti-phospho ATR Ser428 (CST), anti-ATR (Bethyl), anti-LC3B (CST), anti-SQSTM1/p62 (CST), anti-carleticulin (CST), anti-pATM Ser1981 (CST), anti-ATM (1B10 Abnova). The utilized secondary antibodies were anti-rabbit and anti-mouse HRP coniugated (BIORAD), anti-rabbit StarBrightBlue700 (Biorad) and Alexa Fluor 790 (Thermo Fisher Scientific), and anti-mouse Alexa Fluor 680 (Thermo Fisher Scientific). Immunoreactive bands were recorded using the enhanced chemiluminescence (Advansta) or fluorescence acquisition by ChemiDoc Touch Imaging System (Bio-Rad). The whole lane normalization (WLN) strategy was adopted in all western blot analyses using a trihalo compound for protein visualization. Acquired images were analyzed by Image Lab software 5.2.1 (Bio-Rad).

### Indirect Immunofluorescence Microscopy

1×105 cells per well were grown on Lab-Tek II chamber slide (Nunc) 8-well slides upon reaching 70-80% of confluence. After bleomycin treatment for phospho H2AX detection, and for HDAC4 detection, cells were fixed with 4% formaldehyde for 10 minutes and then with 100% cold methanol for 10 minutes. They were subsequently permeabilized with 0.5% NP-40 in PBS for another 10 minutes. After performing the blocking procedure for 1 hour at room temperature, primary antibodies were applied in 0.1% Triton X100, 1% BSA in PBS overnight at 4° C. The following antibodies were used: anti-phospho H2AX Ser139 (GeneTex and Cell Signaling Technology) and anti-HDAC4 (Cell Signaling Technology and Thermo Fisher Scientific). The following day, slides were incubated with secondary anti-mouse TRITC-conjugated antibody (Sigma-Aldrich) or anti-rabbit FITC-conjugated antibody (Sigma-Aldrich) in 0.1% Triton X100, 1% BSA in PBS for 1 hour at 37° C. After washing procedures, DNA was stained with 4′,6-diamidino-2-phenylindole (DAPI) at a final concentration of 0.2 μg/mL. Washed slides were mounted and embedded with ProLong Antifade (Thermo Fisher Scientific). Slides were observed by Olympus IX51, and the images were acquired by ToupCam camera (ToupTek Europe). Image analyses were performed by ImageJ (NIH) (developed at the U.S. National Institutes of Health and available on the Internet at http://rsb.info.nih.gov/nih-image), and H2AX foci numbers were indirectly calculated (after system calibration) by nuclear signal skewness data.

### DCFH-DA Cellular Assay

Cells were seeded in black 96-well plates (6000 cells/well), and 24 h later intracellular ROS levels were examined using a non-fluorescent agent 2′,7′-dichlorofluorescin diacetate (DCFH-DA, Sigma-Aldrich, Milan, Italy), as previously reported by Benedetti et al. (The antioxidant protection of CELLFOOD against oxidative damage in vitro. Food and chemical toxicology: an international journal published for the British Industrial Biological Research Association 2011, 49(9):2292-2298.) on pag. 2294, paragraph 2.6 with slight modifications. Cells were incubated with DCFH-DA (5 μM) at 37° C. for 30 minutes, then the excess probe was removed by washing cells with PBS. DCF oxidation kinetic was detected both at basal condition for 30 minutes, and after the addition of H₂O₂ (100 μM) for additional 30 minutes, monitoring the fluorescence signal in the same 96-well plates.

The fluorescence emission of the probe was measured at 520 nm upon excitation at 485 nm in a FluoStar Optima spectrofluorimeter (BMG Labtech, Offenburg, 223 Germany).

### Mitotracker Red CMX-ROS Assay

Mitochondrial membrane potential was detected by staining live-cells using the Mitotracker Red CMX-ROS as reported in Poot M. et al. (Analysis of mitochondria) morphology and function with novel fixable fluorescent stains. J Histochem Cytochem 1996, 44(12):1363-1372.) on pag. 162 first paragraph, and pag. 163 second paragraph. Cells were seeded in black 96-well plates (6000 cells/well) and 24 h later cells were incubated with the probe (100 nM) diluted in serum-free medium for 18 min (previously verified as non-saturating endpoint), monitoring the dye entry kinetic measuring the emission fluorescence of the probe at 612 nm upon excitation at 584 nm by a FluoStar Optima spectrofluorimeter (BMG Labtech, Offenburg, 223 Germany).

### Mitochondrial DNA Quantification in the Cytoplasm

The release of mtDNA in the cytoplasm has been performed as reported by Yang et al. (NAD(+) supplementation prevents STING-induced senescence in ataxia telangiectasia by improving mitophagy. Aging Cell 2021, 20(4):e13329.) in the supplemental material pag. 6 paragraph: Subcellular fractionation. The amount of mtDNA was quantified performing a quantitative PCR by using primers amplifying NADH-ubiquinone oxidoreductase chain 1 (ND1) DNA as reported in Potenza et al. (Effects of oxidative stress on mitochondrial content and integrity of human anastomotic colorectal dehiscence: a preliminary DNA study. Can J Gastroenterol 2011, 25(8):433-439.) on pag. 436 first paragraph and pag. 435 paragraph Real-time PCR, a specific mitochondrial gene from 5 ng of the cytoplasmic DNA fraction. The Proteasome 20S Subunit Beta 5 (PSMB5) nuclear reference gene was quantified by using the primers: forward 5′-ACGTGGACAGTGAAGGGAAC-3′ and reverse 5′-CTGCTCCACTTCCAGGTCAT-3′, from 2 ng of the nuclear DNA fraction. PCR reactions were performed on a QuantStudio™ 5 Real-Time PCR System and amplification plots were analysed using the QuantStudio™ sequence detection system (Applied Biosystems) and the relative expression data were calculated by the ½^{ΔCt} method.

### NAD+ Assay Quantification

For the NAD+ quantitation, metabolites were extracted from cell pellets (6×106) in ice-cold lysis buffer 6:4 MeOH:ACN, 0.1% formic acid; samples were vortexed and incubated at −20° C. for sample deproteinization. After centrifugation the supernatants were collected and freeze-dried.

### Liquid Chromatography and Mass Spectrometry

The samples were resuspended with 50:30:20 MeOH:ACN:H₂O with 0.1% formic acid for the injection in an UHPLC Vanquish system (Thermo Fisher scientific) coupled to an orbitrap Exploris 240 mass spectrometer. The metabolites separation was performed by an Accucore 150 amide HILIC column (held at 60° C.) and the mobile phases consisted in the phase A, water with 0.1% formic acid and B acetonitrile with 0.1% formic acid both containing 5 mM ammonium formiate. Elution gradiant was 99% of B up to 3 minutes, 1% B in 11 minutes, 1% B for 4 minutes 99% B in 0.2 minutes and 99% B up to 22 minutes. Mass spectrometer, equipped with H-ESI source, operated in positive mode with a scan range 80-800 m/z in DDA manner. Deep scanning strategy was adopted by AcquireX and NAD+ identification and quantitation was performed by Compound Discoverer 3.2 (Thermo Fischer scientific).

### Structural Design of ATM Variants

ATM variants 3D structure have been built up using UCSF Chimera compiled in Linux 64-bit environment.

### Statistical Analysis

GraphPad Prism was used for statistical analyses and graph generation. Statistical tests were chosen according to sample size and variance homogeneity. The statistical tests used in more than two groups comparison were: Friedman test followed by Dunn's test for WB experiments and NAD+ metabolite assay; Kruskal-Wallis test followed by Dunn's test for IF, DCF and mtDNA assays; Welch's ANOVA test for foci analyses and Mitotracker assay. Mann-Whitney test has been used to compare two unpaired samples, while t test for unpaired measures has been applied when data were normally distributed. Means or medians were considered statistically different when p≤0.05.

### RESULTS

### Lentiviral Constructs Assay

The transduced AT 648 hT fibroblasts, by lentiviral system, were tested for the expression of the cloned ATM variants. The expression was verified by Western blotting (WB) using antibody anti-ATM targeting C-terminal domain (clone 1B10) and FIG. 1 shows the ATM 3-52, ATM 4-53, ATM SINT and miniATM transduced AT 648 hT cell lines. The UTD AT 648 hT was also probed as control. The full-length native ATM was also noticed in all the tested cell lines.

### ATM Variants Activity in DSBs

Biochemical properties of the investigated ATM variants were evaluated by testing the canonical ATM targets after bleomycin treatment, which is a radiomimetic agent that causes double strand breaks in DNA, similar to those obtained with radiotherapy. Among ATM substrates, the Ser139 phosphorylated histone H2AX, which is surrounding the DNA break site, acts as an anchor for proteins recruiting, amplifying the DNA damage signal. This mechanism ensures that broken DNA ends do not separate and do not give rise to aberrant rejoining of DNA fragments and chromatin translocations. H2AX is considered the immediate sensor of DSBs and therefore is necessary for the identification and repair of DSBs. Accordingly, yH2AX was detected by indirect immunofluorescence (IF) assay with different approaches to quantify the DNA damage: measuring fluorescence intensity (FIG. 2) and analyzing the quality of foci distribution (FIG. 3) (Lobrich, M. et al. Gampaha foci analysis for monitoring DNA double-strand break repair: strengths, limitations and optimization. Cell cycle 9, 662-669, doi:10.4161/cc.9.4.10764 (2010).; Lu, T. et al. Cellular responses and gene expression profile changes due to bleomycin-induced DNA damage in human fibroblasts in space. PloS one 12, e0170358, doi:10.1371/journal.pone.0170358 (2017). The immunofluorescence detection of yH2AX is considered as the most appropriate method because phosphorylated H2AX forms nuclear foci at the sites of DSBs, and the foci analysis permit to verify the DSBs repair efficiency. If the repair occurs H2AX will progressively dephosphorylate, while its persistent phosphorylation reveals DNA injuries with unrepaired DSBs. As reported in FIG. 2, in all WT hT cells and AT 648 hT transduced cells there was an increase in yH2AX content after 3 h drug treatment compared to the control cell line, indicating that the expressed ATM variants were correctly activated in response to DNA damage, while a significant decrement of yH2AX was observable 24 h post treatment, suggesting that ATM variants could have an influence in DSBs repair defects. Surprisingly, also the analysis of yH2AX in AT 648 hT untransduced cells showed the same pattern of transduced AT fibroblasts. H2AX phosphorylation assay performed by western blot showed the same behavior observed by IF for all treated cells, as reported in FIG. 13 However, the western blotting technique is only useful to monitor the total yH2AX amount, while for individual nuclei the IF method is required.

Once established that ATM variants were proper activated upon bleomycin treatment, we proceeded to estimate the quality of foci distribution, as reported by Lu, T. et al., pointing out the priority on evaluating the foci number rather than signal intensity to monitor DSBs repair process. We in turn, allocated the fluorescence signal of H2AX phosphorylation into three categories in function of signal skewness (FIG. 3A): Type I: Skew<0.05 (pan-nuclear staining), Type II: 0.05≤Skew≤2 (indistinct foci) and Type III: Skew>2 (distinct countable foci). FIG. 14 shows staining types of each tested cell line after the applied treatment. Foci number was obtained from the Type III stain pattern (FIG. 3B). DNA lesions correction depends on foci number reduction. As indicated in FIG. 14, all the AT 648 hT transduced cells presented an increment of Type I stain pattern after 3h drug treatment compared to the untreated AT 648 hT cells. The analysis of foci number revealed that ATM SINT is the most efficient ATM variant in repairing lesions, as demonstrating by the reduced number of Type III foci over a period of 24 h. On the contrary the other ATM variants (ATM 3-52, ATM 4-53) were not able to statistically decrease the foci number in the same lapse of time. MiniATM exhibited a significant diminished number of foci at 24 h post treatment, but it is the least capable in phosphorylating H2AX (FIG. 2, Welch test). The observed amount of yH2AX in miniATM is probably not due to the total average signal derived from both accumulated yH2AX and the meanwhile repair of foci, because miniATM transduced cells displayed elevated foci number over 3 h of bleomycin treatment. Treated WT hT cell lines coherently increased Type I over a period of 3 h, since ATM correctly phosphorylates H2AX, and presented a significant loss of foci number 24 h post bleomycin treatment, suggesting that most foci are solving, and the lesions are reduced. The untransduced AT 648 hT fibroblasts kept high number of foci independently of bleomycin treatment, indicating a persistence of unrepaired DNA lesions, even though the IF fluorescence intensity statistically decreased 24 h post drug. Additionally, a slight increase in Type I foci after 3 h of bleomycin treatment was also noticed in the UTD control cell line AT 648 hT (FIG. 14).

### Untransduced AT 648 hT Cells are Influenced by ATR

It is known that H2AX is also phosphorylated by ATR in response to replication stress. For this reason, we decided to investigate about phosphorylated (Ser428) and total ATR by Western Blotting in all the tested cell lines with or without bleomycin treatment (FIG. 4). ATR when active is autophosphorylated at Ser428, Ser435 and Thr1989. We could not report the ratio p-ATR/ATR, since it did not reflect the real content of these proteins in the tested cell lines because both total and phosphorylated ATR changed upon treatment.

In UTD AT 648 hT cells, p-ATR/WLN ratio resulted constantly elevated, independently of bleomycin treatment. This behaviour could be due to the lack of ATM that in turn, leads to a replication stress condition in AT 648 hT cells, with over phosphorylation of ATR that can phosphorylate H2AX in this cell line as previously reported.

### ATM Variants Activity on ATM Downstream Targets

To further survey the ATM variants biological functionality, some other ATM downstream substrates were tested. The Checkpoint kinase 2 (CHK2) is activated by ATM in response to DNA DSBs, promoting CHK2 dimerization and autophosphorylation, that in turn leads to complete active monomers. Accordingly, we examined the ability of ATM variants to activate this checkpoint kinase by analyzing Thr68 phosphorylation after exposure to bleomycin through Western Blot (FIG. 5). CHK2 phosphorylation is increased after 3 h of bleomycin treatment in all ATM variant transduced cells, while it seemed to decrease 24 h post drug only in ATM 3-52 and ATM SINT transduced cells, with the same behavior of WT hT cells, despite not in a statistically significant way. p-CHK2 is incapable to become entirely activated in ATM variant transduced cell lines, since the only Thr68 phosphorylation persisted over a period of 24 h. Nevertheless, Thr68 phosphorylation is a biomarker of ATM pathway, and we can suppose that all ATM variants were able to activate CHK2. Similarly, AT untransduced fibroblast cells presented p-CHK2, probably due to the presence of ATR activity and the crosstalk signaling between ATM/ATR pathways. Total CHK2 is not reported since it did not change its expression and did not affect the ratio p-CHK2/WLN (data not shown). An additional ATM downstream target is the tumor suppressor p53, that once phosphorylated becomes stabilized and promotes its transcriptional activity. p53 can induce cell cycle arrest ensuring DNA repair through the upregulation of p21, or it can trigger apoptosis when DNA damages are excessive. The phosphorylation of p53 by ATM at Ser15 was assayed by Western Blot (FIG. 6), and the ratio p-p53/p53 was found to be enhanced only in ATM 3-52 after 3 h of bleomycin addition. All transduced AT 648 hT and WT hT cells boosted p-p53 quantity after 24 h of recovery.

The activation of p53 pathway might be slower, since p53 is also a downstream target of CHK2 kinase on Ser20, and this phosphorylation is still required 24 h post drug for the transcription of p21, that is critical to support rather than start Gl/S and G2/M cell cycle arrest.

### Autophagy is Impacted in AT 648 hT Transduced Cells

It has recently been ascertained that ATM owns extra-nuclear functions, since it has 35 been localized in cytoplasmic organelles, including lysosomes, mitochondria, peroxisomes, and synaptic vesicle, also exerting a role in the autophagy, mitophagy and pexophagy pathways. Since it is known that AT cells have an impairment in the autophagy process, particularly in the fusion between autophagosomes and lysosomes, we decided to investigate the autophagic flux in ATM variants transduced cells. Microtubule-associated protein light chain 3 (LC3) expression was used as an autophagy marker to detect autophagic flux progression, because the LC3B-II lipidated form, converted from LC3B-I during autophagy, becomes associated with autophagosomes and autolysosomes, and its amount correlates with the number of autophagosomes. Hence, we performed a WB using antibody anti-LC3B (FIG. 7 In AT transduced cells, at basal condition the LC3B II/I ratio resulted elevated compared to UTD AT 648 hT cells, likely because there is a greater I to II transition, thus improving autophagy.

Autophagy flux improvement by ATM variants in AT cells was also confirmed by the Sequestosome 1 (SQSTM1/p62) analysis, evaluated by WB (FIG. 8), whose degradation reveals a progression of the autophagic process. In basal conditions, p62 protein was elevated in untransduced AT 648 hT cells compared to cells expressing the ATM variants, and to WT hT cells. Accordingly, the p62 accumulation indicated that autophagosomes are not degraded in untransduced AT.

Autophagy also plays a key role in preserving the cellular proteostasis when endoplasmic reticulum (ER) stress occurs, leading to damage proteins removal. A recent work from Poletto and colleagues showed the elevated quantities of oxidized proteins in AT cells with an enhanced nuclear proteosome activity, suggesting a proteostatic stress. Additionally, proteins aggregation was also found in AT cells due to the absence of ATM activation in response to ROS, in agreement with the aberrant protein homeostasis and oxidation in other neurodegenerative diseases. Calreticulin (CARL), a quality control chaperone, is induced under ER stress and could couple ER stress to autophagy, interacting with LC3B. CALR-LC3B complex seems to colocalize in autophagosome, triggers autophagy flux, as a new mechanism of negative feedback to alleviate ER stress. The amount of CALR was detected by WB assay in WT hT and AT 648 hT transduced and untransduced cells (FIG. 9). At basal condition, untransduced AT hT cells showed a CARL overexpression compared to cells expressing ATM variants, indicator of ER stress in AT cells rescued by ATM variants.

The analysis of LC3B, p62 and CALR outcomes endorsed the positive effects of ATM 4-53 and ATM SINT in inducing autophagy flux and alleviating proteostatic stress.

### ATM 4-53 Improve the Scavenger Activity in Counteracting ROS Production

Guo et al. reported that ATM is also triggered by oxidative stress, independently of the DNA DSBs activation confirmed by its presence in the extra-nuclear compartments. Studies reported that AT phenotype is not only due to a defect in DNA-DSB response, but also to a diminished control of ROS, reviewed by Ditch et al. and Watters et al., in fact it was found that ATM-deficient cells are in a constant state of oxidative stress with higher levels of ROS. Additionally, the plasma of AT patients showed a reduced antioxidant capacity and reduced levels of retinol, ubiquinol, and a-tocopherol. ATM-mice, even though they did not develop neurodegeneration, presented boosted oxidative stress markers in restricted areas of the brain. Based on this evidence, we aimed to investigate the antioxidant capacity in ATM variants-transduced cells. Intracellular ROS levels were examined using the 2′-7′dichlorofluorescin diacetate (DCFH-DA) at basal condition (FIG. 10A,) and with the addition of an oxidative stimulus (H₂O₂) (FIG. 10B) monitoring DCF oxidation kinetic. We confirmed the higher level of ROS in AT 648 hT cells compared to the WT hT ones, both in basal and treated conditions, while only the cells expressing the ATM 4-53 and miniATM showed a lower amount of intracellular ROS, indicating a greater antioxidant capacity. The other ATM variants did not show statistically significant differences in counteracting the presence of ROS.

### Mitochondria Dysfunctions are Restored by ATM Variants

Mitochondria are the major source of ROS production through oxidative phosphorylation, and it has been reported that ATM is implicated in maintaining mitochondrial homeostasis. ATM is indeed, involved in the transcription of genes required for mitochondrial function and mtDNA homeostasis, particularly in the brain, which has a high energy request, avoiding a decrease in neurons energy production and consequently neuronal death. Additionally, ATM protein is promptly triggered by mitochondrial dysfunctions promoting mitophagy of aberrant and depolarized mitochondria. Consequently, ATM loss leads to an increase in mitochondrial reactive oxygen species, impaired cellular respiratory capacity, and high mitochondrial mass with decreased membrane potential, due to diminished mitophagy rather than an increase in mitochondrial biogenesis. The mitochondrial dysfunctions and the effects of ATM variants in AT 648 hT cells were tested. Mitochondrial membrane potential was indirectly evaluated by staining cells with MitoTracker Red CMX-ROS, whose entry depends on membrane potential, thus evaluating the dye entry kinetics (FIG. 11A), independently of mitochondria number. As expected, AT 648 hT cells showed a lower mitochondrial functionality compared to WT hT cells, while the cells transduced with the ATM 3-52, ATM 4-53 and ATM SINT presented a faster dye entry, indicating a better mitochondria functionality than the control AT 648 hT cells. miniATM variant was not statistically significantly different than the control.

Yang et al. reported that the classical senescence and inflammatory phenotype of AT cells might be triggered by the release of mtDNA in the cytoplasm from damaged mitochondria that are not properly removed. Aberrant mitophagy has been ascribed to ATM deficiency that in turn, causes NAD+ depletion. In fact, low levels of NAD+, an important metabolite related to mitochondria functionality and co-factor of Sirtuin 1 (SIRT1), are linked to the impaired deacetylation activity of SIRT1, essential for mitochondria biogenesis and chromatin deacetylation. Supplementation with nicotinamide riboside that restored NAD+ levels, improved mitophagy in AT rat primary neurons and AT fibroblasts, rescuing neuropathologic defects and increasing lifespan in AT mice. We in turn, first quantified the amount of mtDNA in the cytoplasmic fraction (FIG. 11B) and secondly, we have quantified NAD+ metabolite (FIG. 11C). Consistent with the study of Yang et al., we found an accumulation of mtDNA in the cytoplasm of UTD AT 648 hT in comparison with WT hT cells. All ATM variants transduced cells statistically decremented the amount of mtDNA in the cytosol. Coherently, a lower amount of NAD+ was observed in AT 648 hT cells than healthy cells, while all ATM variants were able to reinstate NAD+ level.

The results obtained by evaluating mitochondria functionality demonstrated that some of the tested ATM variants transduced in AT cells can rescue mitochondrial AT phenotype.

### The Expression of ATM Variants in AT Cells Can Alter HDAC4 Localization

Based on previous studies about HDAC4 dynamic in AT cells, showing that ATM deficiency led to a nuclear HDAC4 accumulation, promoting neurodegeneration in AT neurons, we decided to investigate the nucleus/cytosol HDAC4 shuttle in the stated cellular model. Performing indirect immunofluorescence (FIG. 12), we found out an accumulation of HDAC4 in untransduced AT 648 hT cell line compared to WT hT cells, as previously published. The nuclear accumulation was described as HDAC4 nuclear/cytosol and nuclear/total ratios. All the cells transduced with all ATM variants showed a reduced significant nuclear accumulation pattern, indicating they were able to modulate HDAC4 shuttle.

### Conclusion

ATM protein variants containing additional domains rather miniATM, are able to rescue ATM phenotype and could be used in gene therapy/gene delivery or mRNA delivery.

Table 2 below summarises the reported experiments and the results obtained showing the overall enhanced effectiveness in restoring ATM functions of ATM SINT when compared to the other variants tested.

TABLE 2

| | | | | | | Oxidative | | |
|---|---|---|---|---|---|---|---|---|
| | | | | Autophagy | ER Stress | stress | Mitochondria | HDAC4 |
| | yH2AX | p-CHK2 | p-p53 | recovery | recovery | recovery | recovery | Shuttle |
| WT | ++ | ++ | ++ | ++ | ++ | ++ | ++ | ++ |
| UTD | −ATR | −ATR | −ATR | − | − | − | − | − |
| | crosstalk | crosstalk | crosstalk | | | | | |
| TD 3-52 | +− | + | + | + | ++ | − | + | + |
| TD 4-53 | +− | +− | + | + | ++ | + | + | + |
| TD SINT | ++ | + | + | + | ++ | − | ++ | ++ |
| TD | +− | +− | + | +− | ++ | ++ | +− | + |
| miniATM | | | | | | | | |

yH2AX: recognition and repair of DNA DSBs.
p-CHK2/p-p53: biomarker of ATM activity in response to DNA damage, involved in the cell cycle arrest ensuring that the DNA is repaired before cell cycle progression.
Autophagy recovery: autophagy progression. This pathway is impaired in A-T cells.
Oxidative stress recovery: antioxidant capacity, ability to counteract ROS production.
Mitochondria recovery: less depolarized damaged mitochondria probably due to a better mitochondria functionality or to a greater mitophagy flux.
HDAC4 Shuttle: capacity to alter HDAC4 localization promoting HDAC4 nuclear export, which is one of the protective mechanisms against neurodegeneration.

1. A variant of the human ATM protein having SEQ ID NO 1, or a derivative thereof.

2. The derivative of the variant according to claim 1, wherein said derivative is characterized in that it further comprises from 3 to 170, or 3 to 135, or 3 to 110, or 3 to 80, or 3 to 65 or 3 to 40 additional amino acids between the amino acids in position 205 and 206 of SEQ ID NO 1.

3. The derivative according to claim 2, wherein said additional amino acids code for one or more phosphorylation domain and are selected from the human ATM protein having SEQ ID NO 5.

4. The derivative according to claim 3, wherein said additional one or more phosphorylation domain is a domain comprising Serine (S) in position 1981 of SEQ ID NO 5, or is a domain comprising Serine (S) in position 1893 of SEQ ID NO 5, or is a domain comprising Serine (S) in position 367 of SEQ ID NO 5, or is a domain comprising Serine (S) in position 794 of SEQ ID NO 5, or is a domain comprising Serine (S) in position 1403 of SEQ ID NO 5.

5. The derivative according to claim 3, wherein said phosphorylation domain is selected from SEQ ID Nos 6-10.

6. A medicament comprising the variant or derivative thereof according to claim 1.

7. A method for preventing, treating or adjuvating the treatment of a disease related to at least one mutation of the ATM gene/s in a subject comprising administering to the subject the medicament of claim 6.

8. The method according to claim 7, wherein said disease related to at least one mutation of the ATM gene/s is Ataxia Telangiectasia or cancer.

9. The method of claim 7, wherein said variant and/or derivative thereof is administered in a therapeutically effective amount to a patient in need thereof, optionally in combination with at least one variant of the human ATM protein having SEQ ID NO 2, SEQ ID NO 3 or SEQ ID NO 4 and/or further therapeutically active compound or drug or drug cocktail or in association to a chemical, radiological or immunological cancer therapy.

10. A nucleotide sequence coding for the variant of the human ATM protein having SEQ ID NO 1 or a derivative thereof, as defined claim 1.

11. The nucleotide sequence according to claim 10 wherein said sequence comprises optimised codons.

12. The nucleotide sequence according to claim 11, wherein said nucleotide sequence is a cDNA or an mRNA.

13. The mRNA according to claim 12 wherein said mRNA comprises a 3′ and a 5′ UTR element flanking the coding sequence, a 5′ Cap and a polyA tail.

14. The mRNA according to claim 12 wherein said mRNA comprises one or more modified nucleosides.

15. The mRNA according to claim 12, wherein said mRNA is complexed with one or more carrier molecules.

16. The mRNA according to claim 15 wherein said mRNA is complexed in a cationic nanoemulsion, in a nanoparticle, in a liposome, in a cationic polymer liposome, in a polysaccharide particle, in a cationic lipid nanoparticle, in a cationic lipid cholesterol nanoparticle, in a cationic lipid cholesterol PEG nanoparticle.

17. A medicament comprising the nucleotide sequence according to claim 10.

18. A method for preventing or treating or adjuvating the treatment of a disease related to at least one mutation of the ATM gene/s in a subject comprising administering to the subject the medicament of claim 17.

19. The method claim 18, wherein said disease related to at least one mutation of the ATM gene is Ataxia Telangiectasia or cancer.

20. An expression vector, comprising a nucleotide sequence according to claim 10 operably linked to a promoter.

21. The expression vector of claim 20 wherein said nucleotide sequence is a cDNA or an RNA.

22. The expression vector of claim 20, wherein said expression vector is selected from a plasmid, a yeast vector, a mammalian vector, a viral vector, a gene therapy expression vector, a single-stranded phage, a double-stranded phage, artificial chromosome.

23. The expression vector according to claim 22, wherein said expression vector is selected from the following list: adenovirus, adeno-associated virus (AAV), lentivirus, retrovirus, cytomegalovirus (CMV), Herpes Simplex Virus (HSV).

24. A medicament comprising the expression vector according to claim 20.

25. A method for preventing or treating or in adjuvating the treatment of a disease related to at least one mutation of the ATM gene/s in a subject comprising administering the medicament of claim 24.

26. The method according to claim 25, wherein said disease related to at least one mutation of the ATM gene is Ataxia Telangiectasia or cancer.

27. A pharmaceutical composition comprising the variant of the human ATM protein or a derivative thereof according to claim 1, and a pharmaceutically acceptable carrier and/or excipient.

28. The pharmaceutical composition according to claim 27 further comprising one or more variant of the human ATM protein having SEQ ID NO 2, 3 or 4, or a nucleotide sequence, a mRNA or an expression vector coding for said one or more variant having SEQ ID NO 2, 3 or 4.

29. The pharmaceutical composition according to claim 27 in a form suitable for administration by systemic injection, central nervous system delivery, aerosol/nasal delivery, topical delivery, RBCs or vesicles.

30. The pharmaceutical composition according to claim 29, for intravenous injection administration, intraparenchymal administration in particular areas of the brain such as intracerebroventricular, cisternal, lumbar or intrathecal administration, or intra-arterial injection administration, or for direct administration into the cerebrospinal fluid.

31. A combination of the variant of the human ATM protein or a derivative thereof according to claim 1 and a drug cocktail or at least one additional therapeutically active compound or drug.

32. A pharmaceutical composition comprising the combination according to claim 31.

33. A method for preventing or treating, or adjuvating the treatment of a disease related to at least one mutation of the ATM gene/s in a subject comprising administering to the subject the pharmaceutical composition of claim 32.

34. The method of claim 33, wherein said disease related to at least one mutation of the ATM gene is Ataxia Telangiectasia or cancer.


SEQUENCE LISTING (10 sequences)

SEQ ID NO: 1 - protein, 935 aa, Homo sapiens
MSLVLNDLLICCRQLEHDRATERKKEVEKFKRLIRDPETIKHLDRHSDSKQGKYLNWDAVFRFLQKYIQKETECLRIAKPNVSASTQASRQKKMQEISSLVKYFIKCANRRAPRLKCQELLNYIMDTVKDSSNGAIYGADCSNILLKDILSVRKYWCEISQQQWLTFGMASHLDYLVLEWLNLQDTEYNLSSFPFILLNYRSCYKVWCPELEELHYGTSYHESLYNALQSLRDREFSTFYESLKYARVKEVEEMCKRSLESVYSLYPTLSRLQAIGELESIGELFSRSVTHRQLSEVYIKWQKHSQLLKDSDFSFQEPIMALRTVILEILMEKEMDNSQRECIKDILTKHLVELSILARTFKNTQLPERAIFQIKQYNSVSCGVSEWQLEEAQVFWAKKEQSLALSILKQMIKKLDASCAANNPSLKLTYTECLRVCGNWLAETCLENPAVIMQTYLEKAVEVAGNYDGESSDELRNGKMKAFLSLARFSDTQYQRIENYMKSSEFENKQALLKRAKEEVGLLREHKIQTNRYTVKVQRELELDELALRALKEDRKRFLCKAVENYINCLLSGEEHDMWVFRLCSLWLENSGQLVKGRDDLRQDAVMQQVFQMCNTLLQRNTETRKRKLTICTYKVVPLSQRSGVLEWCTGTVPIGEFLVNNEDGAHKRYRPNDFSAFQCQKKMMEVQKKSFEEKYEVFMDVCQNFQPVFRYFCMEKFLDPAIWFEKRLAYTRSVATSSIVGYILGLGDRHVQNILINEQSAELVHIDLGVAFEQGKILPTPETVPFRLTRDIVDGMGITGVEGVFRRCCEKTMEVMRNSQETLLTIVEVLLYDPLFDWTMNPLKALYLQQRPEDETELHPTLNADDQECKRNLSDIDQSFNKVAERVLMRLQEKLKGVEEGTVLSVGGQVNLLIQQAIDPKNLSRLFPGWKAWV

SEQ ID NO: 2 - protein, 490 aa, Homo sapiens
MYQPQHKPPGRKRCRKSVVWSNTSSNVQTEDRTEAANRIICTIRSRRPQMVRSVEALCDAYIILANLDATQWKTQRKGINIPADQPITKLKNLEDVVVPTMEIKVDHTGEYGNLVTIQSFKAEFRLAGGVNLPKIIDCVGSDGKERRQLVKGRDDLRQDAVMQQVFQMCNTLLQRNTETRKRKLTICTYKVVPLSQRSGVLEWCTGTVPIGEFLVNNEDGAHKRYRPNDFSAFQCQKKMMEVQKKSFEEKYEVFMDVCQNFQPVFRYFCMEKFLDPAIWFEKRLAYTRSVATSSIVGYILGLGDRHVQNILINEQSAELVHIDLGVAFEQGKILPTPETVPFRLTRDIVDGMGITGVEGVFRRCCEKTMEVMRNSQETLLTIVEVLLYDPLFDWTMNPLKALYLQQRPEDETELHPTLNADDQECKRNLSDIDQSFNKVAERVLMRLQEKLKGVEEGTVLSVGGQVNLLIQQAIDPKNLSRLFPGWKAWV

SEQ ID NO: 3 - protein, 579 aa, Homo sapiens
MSLVLNDLLICCRQLEHDRATERKKEVEKFKRLIRDPETIKHLDRHSDSKQGKYLNWDAVFRFLQKYIQKETECLRIAKPNVSASTQASRQKKMQEISSLVKYFIKCANRRAPRLKCQELLNYIMDTVKDSSNGAIYGADCSNILLKDILSVRKYWCEISQQQWLEGINIPADQPITKLKNLEDVVVPTMEIKVDHTGEYGNLVTIQSFKAEFRLAGGVNLPKIIDCVGSDGKERRQLVKGRDDLRQDAVMQQVFQMCNTLLQRNTETRKRKLTICTYKVVPLSQRSGVLEWCTGTVPIGEFLVNNEDGAHKRYRPNDFSAFQCQKKMMEVQKKSFEEKYEVFMDVCQNFQPVFRYFCMEKFLDPAIWFEKRLAYTRSVATSSIVGYILGLGDRHVQNILINEQSAELVHIDLGVAFEQGKILPTPETVPFRLTRDIVDGMGITGVEGVFRRCCEKTMEVMRNSQETLLTIVEVLLYDPLFDWTMNPLKALYLQQRPEDETELHPTLNADDQECKRNLSDIDQSFNKVAERVLMRLQEKLKGVEEGTVLSVGGQVNLLIQQAIDPKNLSRLFPGWKAWV

SEQ ID NO: 4 - protein, 252 aa, Homo sapiens [sequence omitted]

SEQ ID NO: 5 - protein, 3056 aa, Homo sapiens
MSLVLNDLLICCRQLEHDRATERKKEVEKFKRLIRDPETIKHLDRHSDSKQGKYLNWDAVFRFLQKYIQKETECLRIAKPNVSASTQASRQKKMQEISSLVKYFIKCANRRAPRLKCQELLNYIMDTVKDSSNGAIYGADCSNILLKDILSVRKYWCEISQQQWLELFSVYFRLYLKPSQDVHRVLVARIIHAVTKGCCSQTDGLNSKFLDFFSKAIQCARQEKSSSGLNHILAALTIFLKTLAVNFRIRVCELGDEILPTLLYIWTQHRLNDSLKEVIIELFQLQIYIHHPKGAKTQEKGAYESTKWRSILYNLYDLLVNEISHIGSRGKYSSGFRNIAVKENLIELMADICHQVFNEDTRSLEISQSYTTTQRESSDYSVPCKRKKIELGWEVIKDHLQKSQNDFDLVPWLQIATQLISKYPASLPNCELSPLLMILSQLLPQQRHGERTPYVLRCLTEVALCQDKRSNLESSQKSDLLKLWNKIWCITFRGISSEQIQAENFGLLGAIIQGSLVEVDREFWKLFTGSACRPSCPAVCCLTLALTTSIVPGTVKMGIEQNMCEVNRSFSLKESIMKWLLFYQLEGDLENSTEVPPILHSNFPHLVLEKILVSLTMKNCKAAMNFFQSVPECEHHQKDKEELSFSEVEELFLQTTFDKMDFLTIVRECGIEKHQSSIGFSVHQNLKESLDRCLLGLSEQLLNNYSSEITNSETLVRCSRLLVGVLGCYCYMGVIAEEEAYKSELFQKAKSLMQCAGESITLFKNKTNEEFRIGSLRNMMQLCTRCLSNCTKKSPNKIASGFFLRLLTSKLMNDIADICKSLASFIKKPFDRGEVESMEDDTNGNLMEVEDQSSMNLFNDYPDSSVSDANEPGESQSTIGAINPLAEEYLSKQDLLFLDMLKFLCLCVTTAQTNTVSFRAADIRRKLLMLIDSSTLEPTKSLHLHMYLMLLKELPGEEYPLPMEDVLELLKPLSNVCSLYRRDQDVCKTILNHVLHVVKNLGQSNMDSENTRDAQGQFLTVIGAFWHLTKERKYIFSVRMALVNCLKTLLEADPYSKWAILNVMGKDFPVNEVFTQFLADNHHQVRMLAAESINRLFQDTKGDSSRLLKALPLKLQQTAFENAYLKAQEGMREMSHSAENPETLDEIYNRKSVLLTLIAVVLSCSPICEKQALFALCKSVKENGLEPHLVKKVLEKVSETFGYRRLEDFMASHLDYLVLEWLNLQDTEYNLSSFPFILLNYTNIEDFYRSCYKVLIPHLVIRSHFDEVKSIANQIQEDWKSLLTDCFPKILVNILPYFAYEGTRDSGMAQQRETATKVYDMLKSENLLGKQIDHLFISNLPEIVVELLMTLHEPANSSASQSTDLCDFSGDLDPAPNPPHFPSHVIKATFAYISNCHKTKLKSILEILSKSPDSYQKILLAICEQAAETNNVYKKHRILKIYHLFVSLLLKDIKSGLGGAWAFVLRDVIYTLIHYINQRPSCIMDVSLRSFSLCCDLLSQVCQTAVTYCKDALENHLHVIVGTLIPLVYEQVEVQKQVLDLLKYLVIDNKDNENLYITIKLLDPFPDHVVFKDLRITQQKIKYSRGPFSLLEEINHFLSVSVYDALPLTRLEGLKDLRRQLELHKDQMVDIMRASQDNPQDGIMVKLVVNLLQLSKMAINHTGEKEVLEAVGSCLGEVGPIDFSTIAIQHSKDASYTKALKLFEDKELQWTFIMLTYLNNTLVEDCVKVRSAAVTCLKNILATKTGHSFWEIYKMTTDPMLAYLQPFRTSRKKFLEVPRFDKENPFEGLDDINLWIPLSENHDIWIKTLTCAFLDSGGTKCEILQLLKPMCEVKTDFCQTVLPYLIHDILLQDTNESWRNLLSTHVQGFFTSCLRHFSQTSRSTTPANLDSESEHFFRCCLDKKSQRTMLAVVDYMRRQKRPSSGTIFNDAFWLDLNYLEVAKVAQSCAAHFTALLYAEIYADKKSMDDQEKRSLAFEEGSQSTTISSLSEKSKEETGISLQDLLLEIYRSIGEPDSLYGCGGGKMLQPITRLRTYEHEAMWGKALVTYDLETAIPSSTRQAGIIQALQNLGLCHILSVYLKGLDYENKDWCPELEELHYQAAWRNMQWDHCTSVSKEVEGTSYHESLYNALQSLRDREFSTFYESLKYARVKEVEEMCKRSLESVYSLYPTLSRLQAIGELESIGELFSRSVTHRQLSEVYIKWQKHSQLLKDSDFSFQEPIMALRTVILEILMEKEMDNSQRECIKDILTKHLVELSILARTFKNTQLPERAIFQIKQYNSVSCGVSEWQLEEAQVFWAKKEQSLALSILKQMIKKLDASCAANNPSLKLTYTECLRVCGNWLAETCLENPAVIMQTYLEKAVEVAGNYDGESSDELRNGKMKAFLSLARFSDTQYQRIENYMKSSEFENKQALLKRAKEEVGLLREHKIQTNRYTVKVQRELELDELALRALKEDRKRFLCKAVENYINCLLSGEEHDMWVFRLCSLWLENSGVSEVNGMMKRDGMKIPTYKFLPLMYQLAARMGTKMMGGLGFHEVLNNLISRISMDHPHHTLFIILALANANRDEFLTKPEVARRSRITKNVPKQSSQLDEDRTEAANRIICTIRSRRPQMVRSVEALCDAYIILANLDATQWKTQRKGINIPADQPITKLKNLEDVVVPTMEIKVDHTGEYGNLVTIQSFKAEFRLAGGVNLPKIIDCVGSDGKERRQLVKGRDDLRQDAVMQQVFQMCNTLLQRNTETRKRKLTICTYKVVPLSQRSGVLEWCTGTVPIGEFLVNNEDGAHKRYRPNDFSAFQCQKKMMEVQKKSFEEKYEVFMDVCQNFQPVFRYFCMEKFLDPAIWFEKRLAYTRSVATSSIVGYILGLGDRHVQNILINEQSAELVHIDLGVAFEQGKILPTPETVPFRLTRDIVDGMGITGVEGVFRRCCEKTMEVMRNSQETLLTIVEVLLYDPLFDWTMNPLKALYLQQRPEDETELHPTLNADDQECKRNLSDIDQSFNKVAERVLMRLQEKLKGVEEGTVLSVGGQVNLLIQQAIDPKNLSRLFPGWKAWV

SEQ ID NO: 6 - protein, 35 aa, Homo sapiens
KSMDDQEKRSLAFEEGSQSTTISSLSEKSKEETGI

SEQ ID NO: 7 - protein, 33 aa, Homo sapiens
ADICHQVFNEDTRSLEISQSYTTTQRESSDYSV

SEQ ID NO: 8 - protein, 36 aa, Homo sapiens
HFSQTSRSTTPANLDSESEHFFRCCLDKKSQRTMLA

SEQ ID NO: 9 - protein, 31 aa, Homo sapiens
LCTRCLSNCTKKSPNKIASGFFLRLLTSKLM

SEQ ID NO: 10 - protein, 32 aa, Homo sapiens
AYISNCHKTKLKSILEILSKLKSILEILSKSP